SEQUENCE LISTING (23 sequences)

SEQ ID NO: 1 - protein, 181 aa, synthetic construct
DKDCEMKRTTLDSPLGKLELSGCEQGLHEIKLLGKGTSAADAVEVPAPAAVLGGPEPLMQATAWLNAYFHQPEAIEEFPVPALHHPVFQQESFTRQVLWKLLKVVKFGEVISYQQLAALAGNPAATAAVKTALSGNPVPILIPCHRVVSSSGAVGGYEGGLAVKEWLLAHEGHRLGKPGLG

SEQ ID NO: 2 - protein, 181 aa, synthetic construct
DKDCEMKRTTLDSPLGKLELSGCEQGLHRIIFLGKGTSAADAVEVPAPAAVLGGPEPLIQATAWLNAYFHQPEAIEEFPVPALHHPVFQQESFTRQVLWKLLKVVKFGEVISESHLAALVGNPAATAAVNTALDGNPVPILIPCHRVVQGDSDVGPYLGGLAVKEWLLAHEGHRLGKPGLG

SEQ ID NO: 3 - protein, 137 aa, Homo sapiens [sequence omitted]

SEQ ID NO: 4 - DNA, 543 bp, synthetic construct
  source 1..543: mol_type = other DNA
gacaaagattgcgaaatgaaacgtaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggt

SEQ ID NO: 5 - DNA, 543 bp, synthetic construct
  source 1..543: mol_type = other DNA
gacaaagactgcgaaatgaagcgcaccaccctggatagccctctgggcaagctggaactgtctgggtgcgaacagggcctgcaccgtatcatcttcctgggcaaaggaacatctgccgccgacgccgtggaagtgcctgccccagccgccgtgctgggcggaccagagccactgatccaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagcgagagccacctggccgccctggtgggcaatcccgccgccaccgccgccgtgaacaccgccctggacggaaatcccgtgcccattctgatcccctgccaccgggtggtgcagggcgacagcgacgtggggccctacctgggcgggctcgccgtgaaagagtggctgctggcccacgagggccacagactgggcaagcctgggctgggt

SEQ ID NO: 6 - DNA, 6154 bp, synthetic construct
  source 1..6154: mol_type = other DNA
ctctttccgtctcaggtcgccgctgcgaagggaggtgagcgttcgtcttcctcgcgtggtcgccatctccggcccgccgcgcactccctctcagtcctctgcctgccttctccccttgcgcctcaagggtcattggcggaccccaacccctccccgtgaagtcgttgtgaattttcaagttattttcccctcactctcattcgccgcagctagtctcggctgcctgaattcggtcccgccgccttaggtcactcagtcccgcctgacaagagttctaggtcgctgtctgccctcaggggtctcccattcacccaagttcccagtcgctctcttcctctgctgtccgagcgtggcccgtggcctaacgctgctttagttctctgtgtctgacgctttccctgtgcccgtttccccgcagccgccgccatgtctgcgcatctgcaatggatggtcgtgcggaactgctccagtttcctgatcaagaggaataagcagacctacagcactgtaagtggggcccggatgcgtggctcctgcgggaggagggtcctgagtctcttgactctgggtcagagggcgggaccttcgcatgtctccgggtcccttattcacgatgccttgtgccgcctccttcccaggagcccaataacttgaaggcccgcaattccttccgctacaacggactgattcaccgcaagactgtgggcgtggagccggcagccgacggcaaaggtgtcgtggtggtcattaagcggagatccggtgagttttgtctggtttgggccagagagcggcccctttcccgggtctgggagctgtgattttttactgtcaggcaggaagagcggtaactgccatcgcggcgggcatccctggcgccagggtgttggtctgggtaccggcttccctctcggccgacttgtcagctctgtgagccgcgcgcgtctgagcccgtgtcctcacctgtaaagtggagaaatgaaaaaggacctgaacttcctcggtggttgttgagagttaaggcacggggttgatgttttcagatgaaattctcaaagcaagtcagggtggggatggatggtttcatcccacaggtgggaagattgaggcacaacaggggaggggccctcgctaccacacttaggaggggacaggctgggttggttgcagccatttgattcccattttgcctgtgtaggttagagaagaggtcttggggaccccactccatacattgtgctgtcaggtgcaggcctttttggggattccctgaatgttcgtgtgagtcgggggtctctaatggaggagtccagcttcacatgtgttcttgacagggctgtattttctttttagagtgagtttttctcaggtccttgattggaactgcctcagagccaagggtccttttactcagtggcagcaacaaacgcagtctgttggctagtgatcctcctgtctcagggacacgtagtccagggagcagccaattgcttggcacttggggaccccgttctggggagtcctgaaagctttcacctcttggattgccgaatacatgggtggcccttcctagactaagggactggcctgagtgaggctgggcctctcagccaagctgatgttgaaccactgctgtggggatgggcctggggttcctgggaagctgttcatacccattgccaggagcgtgggctctggctggacctggatcagatcctaactgaagcggcagctttctggcatgagaaaggagtgttttcatggtggacagaattgggctatgagtgtggcagaagctgtgtcctcagtactccgtgatgacgagtgagcctctgtgaaatggacaggtgggaaaacagctacctgctggcctgcccaggcacccgccacgggcccacgctgctcagcttctcaatgtgagactgtccacacctgcgaggtgtgctaaaggtgcaggttaggtggactgaccccaggacctccctgacccccaaccaggccagcggaagcctgccacctcctatgtgcggaccaccatcaacaagaatgctcgcgccacgctcagcagcatcagacacatgatccgcaagaacaagtaccgccccgacctgcgcatggtgagctggggtttggggatcaggcttggggagactggccagtgctgtggggaagggcctcccactactggttgcaatatgggctggagagggatggattcttgctttcagcctactccccacacccagcattggcctagggggcggcttgtggagtgtatgggctgagccttgctctgctcccccgcccccaggcagccatccgcagggccagcgccatcctgcgcagccagaagcctgtgatggtgaagaggaagcggacccgccccaccaagagctcctgagccccctgcccccagagcaataaagtcagctggctttctcacctgcctcgactgggcctccctttttgaaacgctctggggagctctggccctgtgtgttgtcattcaggccatgtcatcaaaactctgcatgtcaccttgtccatctggaggtgatgtcaatggctggccatgcaggaggggtggggtagctgccttgtccctggtgagggcaagggtcactgtcttcacagaaaaagtttgctgacttgtgattgagacctactgtcccattgtgaggtggcctgaagaatcccagctggggcagtggcttccattcagaagaagaaaggccttttctagcccagaagggtgcaggctgagggctgggccctgggccctggtgctgtagcacggtttggggacttggggtgttcccaagacctgggggacgacagacatcacgggaggaagatgagatgacttttgcatccagggagtgggtgcagccacatttggaggggatgggctttacttgatgcaacctcatctctgagatgggcaacttggtgggtggtggcttataactgtaagggagatggcagccccagggtacagccagcaggcattgagcagccttagcattgtccccctactcccgtcctccaggtgtccccatccctcccctgtctctttgagctggctcttgtcacttaggtctcatctcagtggccgctcctgggccaccctgtcacccaagctttcctgattgcccagccctcttgtttcctttggcctgtttgctccctagtgtttattacagcttgtgaggccaggagtttgagaccatcctaggcaacataatgagacaccgtctctaaaataaaattagctgggtgtggtggtgcaccgcctgtggtcccagctcctcagaggttgagtagaggctgaggtgagcggagcacttgagccaagagtatgaggctgcagtgagcccatgagccccaccactacactccagcctggaagacaccatgacacacagtgaggcctggatggggaaagagtcctgctgttgatcctcacatgtttcctgggcacctaactctgtcagccactgccagggaccaaggatccagcatccatggcacccctggttcctgccatcctggggtacccgattcaaagaaggactctgctccctgtctgagaccacccccggctctgactgagagtaaggggactgtcagggcctcgacttgccattggttggggtcgtacggggctgggagccctgcgttttgaggcagaccactgcccttccgacctcagtcctgtctgctccagtcttgcccagctcgaaggagagcagatctgaccacttgccagcccctgtctgctgtgaattaccatttcctttgtccttcccttagttgggtctattagctcagattgagaggtgttgccttaaaactgagttgggtgacttggtacctgctcaggaccccccgcactgtcccaatcccactcaggcccacctccagctggcctcactccgctggtgacttcgtacctgctcaggagcccccactgtcccagtcccactcaggcccatctctggctggcctcactgcgctgggactccgccttcataaggagagctcactgctcacgttagtagatggccccttctcgtgaggcctctcccctggcacctgcttcagttgtcctccacagcactgatttgcagcccacaagctggcaggtttatctgtctcatgtttgtcttgtgctggtgggcaaggggtttgtctagcacaccagcatataatgagatgcttgatgaatggtgcatattgaatgtataaagcccaccggtcctgagagtttgctcactggagactttctggagatggagtctcgctctgttgcccaggctggcgagtgcaatggcgcgatcttggctcactgcagcctccacctcctgggttcaagcgattctcctgcctcagcctcccgagtagctgggattacaggtgggtgtcaccacacccagctcagtattgtatttttagcagagatggggtttcaccattttgcccaggctggtttggaactcctgacttcaaattacccacctgcctcagcctcccaaagtgctggcattacaggcgctcgaggctttctgatgtggctgctgctgctcagaaggccttgtccttaaccacctccttgcctgccctggaggcttgtgcctctaggccccaccccctgtggagtcctgctggctttctccatccctatctgaatcctccctgctgtgtggcctcccctggtctcatccgtaacacagcccagcttagtgggcctctgttcctgcgggtggccagcctgtctgtgtggctgggctggggaggccacgtctggtatctgaatgctatcggtgggttggggtggaggaaccaggagagggctggagggagggagatggtctcagccccacagagtttggagtcctcagtgtgctgagcaaacgtggagacaccatttccctcctctagacctcatcttggagagagagatgttggatggggccatctattccagctttattcacacaaatcatgtctgttggcctggaaattggaaaaccagttaaaccaaaaacatgatattaagaaaacaggcaggctcaccatagtaaaaatgctgaaagccaaagacaaaattgggagaacaaaagaaaagcgtcttgtcacatacagaaggtccctgataaagttagtagctgccctcatcagaaaccaggcccaggcagtggggacacatccagagtgctgaaagaacctcccccaggtcatcctatccccaagagtgatgcccggcagcattcccagctcagggctaatggttcacggaagccaggaatcaaactgcctgggttccagtcccagctctgccagttatgcccagctgtggggacttgggcagctcgtttagtagcaccgtgcctcagtttcccatatgtaaaaggccattttgagtgcctttcacagccctgcataaggcaggtgtctcagtgttcactgctgtctctccagctcttagtccagtagctgcatggtgagtgagcgtagggcgcaccctggaaggctgccaagcccaaagttgtgcagagcgctggggactccagactccccacagcagcagagactcgggactgaggcatcctctgttcacaggacatgctggcatctactgggtcagggctctgctgctcggtggctgtgcaaccttgggcaagttcctcaacctctctgtgtcttcgtaccctcatctgtaacatgcgtgtcgatagaccctactactcagggttgatgagaagattaaatgtgcaaaacctgcttgactgtgcccacaaatcctgattgtaggaataaattaatgactttttataaatattttgatcagatggactcatgatcacagatgtcttcacatgcctatgactaatttgtacacaaactaatgctcgtgtttcccaagcacctggaagacatgccagatccatgtgcagtaatgcctggtggctccaggtctgccccgccgtcctgtggggctgtgagctttcccagcctcctgcccgtgtttgtgaatatcattctgtcctcagctgcatttccagcccaggctgtttggcgctgcccaggaatggtatcaattcccctgtttctcttgtagccagttactagaataaaatcatctactttaaaa

SEQ ID NO: 7 - DNA, 800 bp, synthetic construct
  source 1..800: mol_type = other DNA
ggctgggcctctcagccaagctgatgttgaaccactgctgtggggatgggcctggggttcctgggaagctgttcatacccattgccaggagcgtgggctctggctggacctggatcagatcctaactgaagcggcagctttctggcatgagaaaggagtgttttcatggtggacagaattgggctatgagtgtggcagaagctgtgtcctcagtactccgtgatgacgagtgagcctctgtgaaatggacaggtgggaaaacagctacctgctggcctgcccaggcacccgccacgggcccacgctgctcagcttctcaatgtgagactgtccacacctgcgaggtgtgctaaaggtgcaggttaggtggactgaccccaggacctccctgacccccaaccaggccagcggaagcctgccacctcctatgtgcggaccaccatcaacaagaatgctcgcgccacgctcagcagcatcagacacatgatccgcaagaacaagtaccgccccgacctgcgcatggtgagctggggtttggggatcaggcttggggagactggccagtgctgtggggaagggcctcccactactggttgcaatatgggctggagagggatggattcttgctttcagcctactccccacacccagcattggcctagggggcggcttgtggagtgtatgggcgccttgctctgctcccccgcccccaggcagccatccgcagggccagcgccatcctgcgcagccagaagcctgtgatggtgaagaggaagcggacccgccccaccaagagctcc

SEQ ID NO: 8 - DNA, 800 bp, synthetic construct
  source 1..800: mol_type = other DNA
gcctccctttttgaaacgctctggggagctctggccctgtgtgttgtcattcaggccatgtcatcaaaactctgcatgtcaccttgtccatctggaggtgatgtcaatggctggccatgcaggaggggtggggtagctgccttgtccctggtgagggcaagggtcactgtcttcacagaaaaagtttgctgacttgtgattgagacctactgtcccattgtgaggtggcctgaagaatcccagctggggcagtggcttccattcagaagaagaaaggccttttctagcccagaagggtgcaggctgagggctgggccctgggccctggtgctgtagcacggtttggggacttggggtgttcccaagacctgggggacgacagacatcacgggaggaagatgagatgacttttgcatccagggagtgggtgcagccacatttggaggggatgggctttacttgatgcaacctcatctctgagatgggcaacttggtgggtggtggcttataactgtaagggagatggcagccccagggtacagccagcaggcattgagcagccttagcattgtccccctactcccgtcctccaggtgtccccatccctcccctgtctctttgagctggctcttgtcacttaggtctcatctcagtggccgctcctgggccaccctgtcacccaagctttcctgattgcccagccctcttgtttcctttggcctgtttgctccctagtgtttattacagcttgtgaggccaggagtttgagaccatcctaggcaacataatgagacaccgtc

SEQ ID NO: 9 - RNA, 19 nt, synthetic construct
  source 1..19: mol_type = other RNA
ctttctcacctgcctcgac

SEQ ID NO: 10 - RNA, 20 nt, synthetic construct
  source 1..20: mol_type = other RNA
accaacgatgtcagagacat

SEQ ID NO: 11 - RNA, 20 nt, synthetic construct
  source 1..20: mol_type = other RNA
cctgtgtttacagatcgctg

SEQ ID NO: 12 - RNA, 20 nt, synthetic construct
  source 1..20: mol_type = other RNA
gggtgatgttgtgcacgtag

SEQ ID NO: 13 - RNA, 20 nt, synthetic construct
  source 1..20: mol_type = other RNA
gtgaagaggactctctgtcg

SEQ ID NO: 14 - RNA, 20 nt, synthetic construct
  source 1..20: mol_type = other RNA
gcttcgatggacattcacgg

SEQ ID NO: 15 - RNA, 20 nt, synthetic construct
  source 1..20: mol_type = other RNA
agacacgcttcactgatacc

SEQ ID NO: 16 - RNA, 20 nt, synthetic construct
  source 1..20: mol_type = other RNA
atgttaacggaactcctgag

SEQ ID NO: 17 - RNA, 20 nt, synthetic construct
  source 1..20: mol_type = other RNA
catcgtcctggacaagctag

SEQ ID NO: 18 - RNA, 20 nt, synthetic construct
  source 1..20: mol_type = other RNA
ataaagtaacactcctctag

SEQ ID NO: 19 - RNA, 20 nt, synthetic construct
  source 1..20: mol_type = other RNA
tcttaccaacccatccaagg

SEQ ID NO: 20 - RNA, 20 nt, synthetic construct
  source 1..20: mol_type = other RNA
aagatctcgctacccgacgg

SEQ ID NO: 21 - DNA, 50 bp, synthetic construct
  source 1..50: mol_type = other DNA
cggaggcccaacctctccgacgacaggtcgccagaggacagcgtgtcagc

SEQ ID NO: 22 - DNA, 28 bp, synthetic construct
  source 1..28: mol_type = other DNA
cctcgccctccgggctccgttaatgatc

SEQ ID NO: 23 - DNA, 25 bp, synthetic construct
  source 1..25: mol_type = other DNA
ctgcgagggaacccccagccgcgca

(19) United States
(12) Patent Application Publication
(10) Pub. No.: US 2026/0347799 A1
(43) Pub. Date: Nov. 5, 2026

(54) METHODS FOR LABELING RIBOSOMES

(71) Applicant: THE BOARD OF REGENTS OF THE UNIVERSITY OF TEXAS SYSTEM, Austin, TX (US)

(72) Inventors: Michael Buszczak, Dallas, TX (US); Chunyang Ni, Dallas, TX (US)

(21) Appl. No.: 19/107,439

(22) PCT Filed: Aug. 30, 2023
(86) PCT No.: PCT/US2023/073188

§ 371 Date: Feb. 27, 2025

Related U.S. Application Data
(60) Provisional application No. 63/402,840, filed on Aug. 31, 2022.

(51) Int. Cl. C12N 9/10; C12N 5/071; C12N 5/0735; C12N 5/074; C12N 5/09; C12N 9/22; C12N 15/10; C12N 15/52; C12N 15/62; C12Q 1/48

(52) U.S. Cl. CPC C12N 9/1007; C12N 5/0606; C12N 5/0686; C12N 5/0693; C12N 5/0696; C12N 9/222; C12N 15/102; C12N 15/52; C12N 15/62; C12Q 1/48; C12Y 201/01063; C12N 2510/04

(57) ABSTRACT

Compositions, methods and kits for labeling ribosomes using a O-6-alkylguanine-DNA alkyltransferase (AGT) analog, variant or derivative, to study the diversity, complexity, dynamics, and regulation of ribosome production and aspects of ribosome function.

### CROSS-REFERENCE TO RELATED APPLICATION

This application claims priority to U.S. Provisional Patent Application Ser. No. 63/402,840, filed Aug. 31, 2022, and titled “METHODS FOR LABELING RIBOSOMES,” the entirety of which is incorporated herein by reference.

### ACKNOWLEDGEMENT OF GOVERNMENT SUPPORT

This invention was made with government support under Grant No. GM125812 awarded by the National Institutes of Health. The government has certain rights in the invention.

### SEQUENCE LISTING

This application contains a Sequence Listing that has been submitted in WIPO ST.26 .xml format via EFS-Web and is hereby incorporated by reference in its entirety. The .xml copy is named 106546-768161 UTSD 3749.xml and is 30 KB in size.

### BACKGROUND

### 1. Field

The present disclosure relates to methods of making and using SNAP-tagged ribosomes. In one aspect, the disclosure relates to cells expressing a fusion polypeptide comprising SNAP-tagged ribosomal proteins

### 2. Background

The homeostatic balance between protein synthesis and turn-over is tightly associated with the growth, health, and longevity of almost all organisms. In multicellular organisms, protein synthesis can vary greatly between different cell types, even in cells from the same lineage. Protein production depends on ribosomes, and changes in normal ribosome levels have important consequences: enhanced ribosome biogenesis often drives tumor growth, while defects in ribosome assembly and function cause a group of diseases collectively known as ribosomopathies, which share several common phenotypes including anemia and a broad range of craniofacial defects. In addition, ribosome malfunction contributes to age-related phenotypes and various neurodegenerative disorders.

Ribosome biogenesis represents a complex process involving multiple steps that must be tightly coordinated with different cellular functions and activities. Ribosome assembly depends on rDNA transcription, rRNA processing, synthesis of 80 ribosomal proteins, assembly of these proteins with 4 different rRNAs, and molecular trafficking between the nucleolus, nucleoplasm, and cytoplasm. Most of what is known about eukaryotic ribosome biogenesis comes from genetic and biochemical studies performed using S. cerevisiae. Although the basic features of ribosome assembly are shared between yeast and humans, ribosome biogenesis has become more complex through evolution (FIGS. 1G and 1H). However, methods to study ribosomal biogenesis in higher eukaryotes are limited.

There is therefore a need in the field to develop methods to study ribosomes in cells from higher eukaryotes including humans.

### SUMMARY

In some aspects, the current disclosure encompasses a fusion polypeptide comprising an amino acid sequence corresponding to a O-6-alkylguanine-DNA alkyltransferase (AGT) analog, variant and/or a derivative thereof, and a ribosomal protein or variant, derivative, or fragment thereof. In some aspects, the ribosomal protein is RPL28. In some aspects, the analog, variant and/or a derivative of AGT is a SNAP-tag or a CLIP-tag. In some aspects, the SNAP-tag or CLIP-tag comprises an amino acid sequence at least 70% identical to SEQ ID NOS: 1 or 2 respectively. In some aspects, the fusion polypeptide comprises an amino acid sequence at least 70% identical to SEQ ID. NO: 3.

In some aspects, the current disclosure also encompasses polynucleotide sequences comprising a nucleic acid sequence encoding a fusion polypeptide provided herein. In some aspects, the polynucleotide sequence comprises a nucleic acid sequence at least about 60% identical to SEQ ID NOS: 4 or 5 and further comprises a sequence at least 60% identical to SEQ ID. NO: 6.

In some aspects, the present disclosure also encompasses engineered host cell comprising a polynucleotide sequence provided herein. In some aspects, the engineered host cell comprises a fusion polypeptide comprising an amino acid sequence corresponding to: an AGT analog, variant and/or a derivative thereof; and a ribosomal protein or variant, derivative, or fragment thereof. In some exemplary aspects of the engineered host cell, the ribosomal protein is RPL28 and the AGT analog, variant and/or a derivative is a SNAP-tag or a CLIP-tag. In some aspects, the SNAP-tag or CLIP-tag comprises an amino acid sequence at least 70% identical to SEQ ID NOS: 1 or 2 respectively and the fusion polypeptide comprises an amino acid sequence at least 70% identical to SEQ ID. NO: 3. In some aspects, the engineered host cell is a somatic cell or a pluripotent cell. In some aspects, the engineered host cell is a human cell. In some aspects, the engineered host cell is selected from, for example, HEK293T, HCT116, iPS or hES cell.

In some aspects the current disclosure also encompasses a method for generating an engineered host cell comprising a SNAP-tagged and/or CLIP-tagged ribosome, the method comprising delivering to a cell: (a) a first site-directed nuclease cutting at a target site within or near a gene that encodes a ribosomal protein; and (b) a nucleic acid comprising a nucleotide sequence encoding a SNAP-tagged and/or CLIP-tagged ribosomal protein that is flanked by (i) a nucleotide sequence homologous with a region located upstream of the target site of (a) and (ii) a nucleotide sequence homologous with a region located downstream of the target site of (a), wherein the first site-directed nuclease cleaves the target site of (a) and the nucleic acid of (b) is inserted at a site that partially overlaps, completely overlaps, or is contained within, the site of (a). In some aspects, the ribosomal protein is RPL28. In some aspects, the site-directed nuclease is a Cas nuclease. In some aspects, the nucleotide sequence homologous with a region located upstream of the target site is at least about 80% identical to SEQ ID NO: 7. In some aspects the nucleotide sequence homologous with a region located downstream of the target site is at least about 80% identical to SEQ ID NO: 8.

In some aspects, the current disclosure also encompasses a method of labeling a ribosome in an engineered host cell, the method comprising: expressing a fusion polypeptide provided herein; and introducing a first substrate molecule in the engineered host cell. In some aspects, the method further comprising introducing a second and/or a third substrate molecule in the cell.

In some aspects, the current disclosure also encompasses a method of heterochronic labeling of one or more ribosomes in an engineered host cell, the method comprising: expressing any fusion polypeptide provided herein in the engineered host cell; introducing a first substrate molecule into the engineered host cell; introducing a second and/or a third substrate molecule in the engineered host cell, wherein the second and third substrate molecule is different from the first substrate molecule. In some aspects, (a) comprises genetically modifying the cell to comprise a polynucleotide sequence encoding the fusion polypeptide. In some aspects, the engineered host cell is a somatic cell or a pluripotent cell. In some aspects, the engineered host cell is a human cell for example a HEK293T, HCT116, iPS, or a hES cell. In some aspects of the method provided herein, the first substrate, the second substrate and the third substrate are cell-permeable substrates. In some aspects the first substrate and/or the second substrate and/or the third substrate is selected from a group consisting of group consisting of BG-GLA-NHS, BG-Maleimide, BG-NH2, BG-PEG-NH2, SNAP-Biotin®, SNAP-Cell® 505-Star, SNAP-Cell® Block, SNAP-Cell® Oregon Green®, SNAP-Cell® TMR-Star, SNAP-Cell® 430, SNAP-Cell® 647-SiR, SNAP-Surface® 488, SNAP-Surface® 549, SNAP-Surface® 594, SNAP-Surface® 649, SNAP-Surface® Alexa Fluor® 488, SNAP-Surface® Alexa Fluor® 546, SNAP-Surface® Alexa Fluor® 647, SNAP-Surface® Block, BG-NH2, CLIP-Biotin®, CLIP-Cell® 505-Star, CLIP-Cell® Block, CLIP-Cell® TMR-Star, CLIP-Surface® 488, CLIP-Surface® 547, CLIP-Surface® 547, CLIP-Surface® 647.

In some aspects, the current disclosure also encompasses a method of visualizing ribosomes in a cell, the method comprising: expressing a fusion polypeptide as provided herein, introducing a first substrate molecule into the cell, wherein the first substrate molecule specifically binds to a first ribosome population; and visualizing the cell using one or more of fluorescence visualization methods. In some aspects, the method further comprises introducing a second or third substrate molecule in the cell. In some aspects, the second or third substrate molecule labels a second or third ribosome population. In some aspects, the first, the second, and the third ribosome population are heterochronic. Non-limiting examples of substrates include BG-GLA-NHS, BG-Maleimide, BG-NH2, BG-PEG-NH2, SNAP-Biotin®, SNAP-Cell® 505-Star, SNAP-Cell® Block, SNAP-Cell® Oregon Green®, SNAP-Cell® TMR-Star, SNAP-Cell® 430, SNAP-Cell® 647-SIR, SNAP-Surface® 488, SNAP-Surface® 549, SNAP-Surface® 594, SNAP-Surface® 649, SNAP-Surface® Alexa Fluor® 488, SNAP-Surface® Alexa Fluor® 546, SNAP-Surface® Alexa Fluor® 647, SNAP-Surface® Block, BG-NH2, CLIP-Biotin®, CLIP-Cell® 505-Star, CLIP-Cell® Block, CLIP-Cell® TMR-Star, CLIP-Surface® 488, CLIP-Surface® 547, CLIP-Surface® 547, CLIP-Surface® 647.

In some aspects, the current disclosure encompasses methods purifying ribosomes from a cell, the method comprising expressing a fusion polypeptide provided herein and further comprising a ribosome purification step. In some aspects ribosome purification step comprises use of a SNAP/CLIP-tag-Trap.

In some aspects, the current disclosure also encompasses a kit comprising a polynucleotide, polypeptide or host cells provided herein and instructions for use. In some aspects the kit further comprises suitable substrates, reagents, buffers, diluents, cells, standards, and containers.

### BRIEF DESCRIPTION OF THE DRAWINGS

Embodiments of the present disclosure are illustrated by way of example in which like reference numerals indicate similar elements and in which:

FIG. 1A is a western blot of control HEK293T cells and cells carrying a SNAP-tag at the endogenous ribosomal protein L28 (RPL28) locus probed with the indicated antibodies.

FIG. 1B shows the distribution of SNAP-tagged RPL28 across a sucrose gradient. The blot shows that the protein co-fractionates with the 60S subunit, 80S monosome, and polysomes as expected. GAPDH is included as a free control. WCL, whole-cell lysate.

FIG. 1C shows the distribution of SNAP-tagged RPL28 from human ESCs across a sucrose gradient.

FIG. 1D are images showing HEK293T cells expressing SNAP-tagged RPL28 were treated with DMSO or unconjugated benzylguanine (SNAP-block) and then labeled with benzylguanine-Oregon green for 30 minutes. Treatment with benzylguanine “blocks” the labeling of pre-existing ribosomes. Scale bars represent 20 μm.

FIG. 1E shows pulse labeling western blots. Wild type HEK293T or HEK293T-RPL28-SNAP cells were pulse-labeled with Methionine, or AHA, or AHA with anisomycin (ANS) for 3 h. Protein extract was labeled with biotin and analyzed by blotting with HRP-Streptavidin. The unfixed total protein staining demonstrates equal loading.

FIG. 1F is a graph showing growth comparison of HEK293T cells. Wildtype HEK293T or HEK293T-RPL28-SNAP cells were seeded at 2.0×10⁵ cells and were counted 24, 48, 72 h after seeding. (n=6 counts per time point). Mean±S.E.M. is shown.

FIG. 1G Heat map of mRNA expression levels of all 80 human ribosomal proteins across the indicated tissues.

FIG. 1H Heat map of mRNA expression levels of ribosome biogenesis factors not found in yeast (Tafforeau et al., 2013).

FIG. 2A shows a schematic detailing the tagging of the RPL28 open reading frame with a SNAP tag at the 3′ end of exon 5 (E5). The SNAP-tagged proteins can be indelibly labeled with a number of different benzylguanine substrates. They can also be “blocked” through the use of unconjugated benzylguanine.

FIG. 2B is an outline of a standard pulse-labeling experiment in which ribosomes older than 48 h are labeled with TMR-Star (red) and ribosomes 0-24 h old are labeled with Oregon green. Cell lysates were subjected to sucrose gradient fractionation. Old and newly labeled RPL28 were incorporated into the 60S, monosome, and polysome fractions.

FIG. 2C show imaging data for pulse-labeled Ribo-SNAP in HEK293T cells. After the final labeling step, the cells were incubated for variable lengths of time. At the start of the experiment, newly labeled RPL28 are clearly observed in the nucleoli of cells, whereas old ribosomes are cytoplasmic, as expected. The nucleolar levels of newly labeled RPL28 continue to drop over the course of 4 h. Scale bar, 10 mm

FIG. 2D show imaging data for pulse-labeled Ribo-SNAP in HCT116 cells After the final labeling step, the cells were incubated for variable lengths of time. At the start of the experiment, newly labeled RPL28 are clearly observed in the nucleoli of cells, whereas old ribosomes are cytoplasmic, as expected. The nucleolar levels of newly labeled RPL28 continue to drop over the course of 4 h. Scale bar, 10 mm

FIG. 2E show imaging data for pulse-labeled Ribo-SNAP in hESC cells. After the final labeling step, the cells were incubated for variable lengths of time. At the start of the experiment, newly labeled RPL28 are clearly observed in the nucleoli of cells, whereas old ribosomes are cytoplasmic, as expected. The nucleolar levels of newly labeled RPL28 continue to drop over the course of 4 h. Scale bar, 10 mm

FIG. 2F is a graph showing the rate at which the levels of nuclear RPL28 decreases varies between HEK293T, HCT116, and hESCs. Values were normalized to the mean of the 0-h group of each cell type. Mean±SD is shown.

FIG. 2G shows images of H9 hESCs carrying the RPL28 SNAP tag reporter or their randomly differentiated progeny were pulse labeled for old and new ribosomes and examined after a 4-h chase. Levels of labeled ribosomes appear relatively homogeneous within hESCs but their differentiated progeny displayed a range of old and new ribosome levels. Moreover, differentiated cells exhibited variable amounts of newly labeled nucleolar RPL28 after a 4-h chase period. Scale bar, 10 mm.

FIG. 2H shows that treatment of cells with the Poll and TOP2B inhibitor CX-5461 results in retention of newly labeled RPL28 within nucleoli after a 4-h chase period. Scale bar, 10 mm

FIG. 2I shows images of cells carrying SNAP-tagged RPL28 transduced with sgRNAs targeting FBL and subjected to pulse labeling. Disruption of FBL results in retention of newly labeled RPL28 within the nuclei after a 4-hr chase period. Scale bar, 20 mm.

FIG. 2J shows the distribution of old and new ribosomes in HEK293T cells treated with NaAsO2. Scale bars represent 20 μm.

FIG. 2K shows 60S subunits present during NaAsO₂ treatment accumulated in the 60S and polysome fractions, whereas ribosomes generated after the treatment are more readily recruited into the polysome fractions.

FIG. 2L Cells carrying SNAP-tagged RPL28 transduced with sgRNAs targeting TSR2 and subjected to pulse-labeling.

FIG. 3A shows a schematic overview of CRISPR-Cas9 screen design.

FIG. 3B shows the MAGeCK analysis data identifying positive hits that result in lower levels of newly labeled RPL28 and increased levels of old ribosomes.

FIG. 3C shows flow cytometry validation of genes identified in the primary screen . . . . Flow cytometry validation of HEK293T cells labeled for new and old ribosomes transduced with sgRNAs targeting the indicated gene (red) compared to control cells receiving an empty vector (black). (n>10,000 cells per condition). The ratio of old to new ribosomes is plotted on each x-axis.

FIG. 3D shows the functional categorization of genes identified in the screen.

FIG. 3E shows imaging data validating representative hits from each of the functional categories using HEK293T cells. Strong retention of newly labeled nucleolar RPL28 after a 4-h chase period distinguishes C1orf109 and SPATA5 from other positive hits. Scale bars, 20 mm.

FIG. 4A provides northern blots for pre-rRNA species showing that disruption of C1orf109 and SPATA5 results in an increase of pre-47S/45S rRNA.

FIG. 4B provides northern blots for pre-rRNA species showing that disruption of C1orf109 and SPATA5 results in a reduction in 12S rRNA in HEK293T cells.

FIG. 4C provides levels of mature 28S and 18S from wildtype and C1orf109^{KO} and SPATA5^{KO} cells.

FIG. 4D provides quantification of the northern blot results with normalized 45S: 18S ratio plotted along the y-axis. n=5 biological replicates. Mean±SEM is shown. p values were calculated using a ratio-paired t test.

and 4E provides quantification of the northern blot results with relative 28S:18S ratio plotted along the y-axis. n=3 biological replicates (right). Mean±SEM is shown. p values were calculated using a ratio-paired t test.

FIG. 4F provides quantification of EU labeling in wild-type, C1orf109^{KO}, and SPATA5^{KO} (n=3 biological replicates), Values were normalized to the mean of wild type. Mean±SEM is shown. p values were calculated by nested one-way ANOVA with Dunnett's multiple comparisons test.

FIG. 4G shows polysome profiles from wild-type, C1orf109^{KO}, and SPATA5^{KO} cells. Red arrows indicate half-mer polysomes, which reflect defective 60S biogenesis.

FIG. 4H is a schematic describing AHA pulse labeling of ongoing protein synthesis.

FIG. 4I shows the total protein as assayed by Ponceau in wild-type (WT) control, C1orf109^{KO}, and SPATA5^{KO} cells.

FIG. 4J shows the total protein as assayed by biotin labeling of AHA-labeled proteins in wild-type (WT) control, C1orf109^{KO}, and SPATA5^{KO} cells.

FIG. 5A shows wild-type (WT) controls and HEK293T cells transduced with sgRNAs targeting C1orf109 and SPATA5 stained for RSL24D1 (green) and DNA (blue). While most RSL24D1 localizes to the nucleoli of control cells, disruption of C1orf109 and SPATA5 results in ectopic localization of RSL24D1 to the cytoplasm. Scale bars, 20 mm.

FIG. 5B shows a schematic overview of SNAP-tagged ribosome pull-down.

FIG. 5C shows a western blot analysis of cytoplasmic extracts and Ribo-SNAP pull-down samples probed with either RSL24D1 or RPL24 antibodies. Loss of C1orf109 and SPATA5 results in a dramatic increase in cytoplasmic localization and ribosome associated RSL24D1.

FIG. 5D shows western blot of sucrose gradient fractions from WT cells probed with antibodies against RPL28 and RSL24D1. Most RSL24D1 was observed in the 60S/80S fraction, with a sharp drop-off in the 80S/light polysome (LP) fraction.

FIG. 5E shows western blot of sucrose gradient fractions from C1orf109^{KO} cells probed with antibodies against RPL28 and RSL24D1.

FIG. 5F shows western blot sucrose gradient fractions from SPATA5^{KO} cells probed with antibodies against RPL28 and RSL24D1.

FIG. 6A shows a schematic of SPATA5 domain structure. The two allelic variants indicated in red have been linked with microcephaly, hearing loss, and intellectual disability in humans.

FIG. 6B shows a schematic describing how the labeling of SNAP-tagged RPL28 was conducted.

FIG. 6C shows images of HEK293T cells transduced with an sgRNA targeting SPATA5, transfected with plasmids carrying transgenes corresponding to wild-type SPATA5 or the disease-linked (Thr330del and D628G) allelic variants, and old ribosomes were pulse labeled with TMR-Star (red) and new RPL28 proteins were labeled with Oregon green (green), as indicated. The wild-type transgene rescued the RPL28 nucleolar retention phenotype caused by loss of SPATA5, whereas cells expressing the two allelic variants continued to display low levels of newly labeled nucleolar RPL28 after a 4-h chase period (yellow arrows).

FIG. 6D shows images confirming that in stained cells (RSL24D1 (green) and DNA (blue)) in contrast to wild-type transgenic controls, expression of the Thr330del and D628G variants did not fully rescue the localization defects of RSL24D1 in SPATA5 mutant cells.

FIG. 6E shows quantitation of Oregon green (OG)-labeled new SNAP-tagged RPL28 within nuclei in the indicated genetic backgrounds. n=3 biological replicates.

FIG. 6F shows quantitation of nuclear RSL24D1 staining in the indicated genetic backgrounds. n=3 biological replicates. Statistical comparisons in (E and F) are between wild-type cells and cells with each of the indicated rescuing transgenes. Mean±SEM is shown. p values were calculated by nested one-way ANOVA with Dunnett's multiple comparisons test.

FIG. 6G shows western blot analysis of cytoplasmic extracts and Ribo-SNAP pull-down samples probed with RSL24D1 and RPL24 antibodies. Wild-type transgenes could partially rescue the increase of cytoplasmic RSL24D1 and its aberrant prolonged association with the 60S subunit in SPATA5 mutant cells. By contrast, the amount of cytoplasmic and ribosome bound RSL24D1 were modestly elevated upon expression of the Thr330del and D628G allelic variants relative to the wild-type control. Scale bars, 10 mm.

FIG. 7A shows string analysis that indicates C1ORF109, SPATA5, SPATA5L1 and CINP likely associate with one another.

FIG. 7B shows immunoprecipitation of FLAG-tagged C1ORF109 from HEK293T cell extracts crosslinked with DSP confirm interactions. Results show that C1ORF109 physically interacts with SPATA5, SPATA5L1, and CINP, but not with RPL28.

FIG. 7C shows pull-down of SNAP-tagged RPL28 showing that SPATA5, SPATA5L1, and CINP interact with ribosomes, but C1ORF109 does not.

FIG. 7D provides images showing that knockout of SPATA5L1 or CINP leads to defects in the trafficking of newly labeled RPL28 from the nucleolus to the cytoplasm.

FIG. 7E shows images of control, SPATA5L^{KO}, and C1orf109^{KO} cells stained for RSL24D1 (green) and DNA (blue).

FIG. 7F shows the matchmaker alignment of CINP and C1ORF109 AlphaFold predicted structures.

FIG. 7G shows the matchmaker alignment of SPATA5 and SPATA5L1 (lower panel).

FIG. 7H imaging data for control, CINP^{KO}, and C1orf109^{KO} cells transfected with a control or C1orf109 rescue construct stained for old and new ribosomes.

FIG. 7I shows western blot analysis showing cytoplasmic lysates from C1ORF109-3xFLAG expressing cells subjected to sucrose gradient fractionation and probed for SPATA5, SPATA5L1, C1ORF109 and CINP.

FIG. 7J shows that SPATA5L1, CINP, and C1ORF109 transgenes do not rescue the ribosome biogenesis defects on SPATA5^{KO} cells. Control and SPATA5^{KO} HEK293T cells transfected with wildtype SPATA5, CINP, C1ORF109 and SPATA5L1 rescue constructs stained for old and new ribosomes according to the schematic. Scale bars represent 20 μm.

FIG. 7K shows a western blot revealing that loss of C1ORF109 or SPATA5 results in decreased levels of SPATA5L1 and CINP.

FIG. 8A provides a schematic model for the function of SPATA5, C1ORF109, SPATA5L1, and CINP.

FIG. 8B provides a schematic model for the function of SPATA5, C1ORF109, SPATA5L1, and CINP mutants.

The drawing figures do not limit the present disclosure to the specific embodiments disclosed and described herein. The drawings are not necessarily to scale, emphasis instead being placed on clearly illustrating principles of certain embodiments of the present disclosure.

### DETAILED DESCRIPTION

The following detailed description references the accompanying drawings that illustrate various embodiments of the present disclosure. The drawings and description are intended to describe aspects and embodiments of the present disclosure in sufficient detail to enable those skilled in the art to practice the present disclosure. Other components can be utilized and changes can be made without departing from the scope of the present disclosure. The following description is, therefore, not to be taken in a limiting sense.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Further, as the present disclosure is susceptible to embodiments of many different forms, it is intended that the present disclosure be considered as an example of the principles of the present disclosure and not intended to limit the present disclosure to the specific embodiments shown and described. Any one of the features of the present disclosure may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present disclosure may include a variety of combinations and/or integrations of the embodiments described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The terms “nucleic acid”, “nucleic acid molecule”, and “polynucleotide” are used interchangeably herein. The terms “nucleic acid encoding . . . ”, or “nucleic acid molecule encoding.” should be understood as referring to the sequence of nucleotides which encodes a polypeptide.

A polynucleotide described herein may comprise one or more nucleic acids each encoding a polypeptide, operably linked to (i.e., in a functional relationship with) one or more regulatory sequences, such as a promoter. Such a polynucleotide may alternatively be referred to herein as a “nucleic acid construct” or “construct”. As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s).

The terms “polypeptide” and “protein,” as used interchangeably herein, refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

An amino acid sequence that is “derived from” an amino acid sequence disclosed herein can refer to an amino acid sequence that differs by one or more amino acids compared to the reference amino acid sequence, for example, containing one or more amino acid insertions, deletions, or substitutions as disclosed herein. The terms “derivative,” “variant,” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide. A part or fragment of a polypeptide may correspond to at least 1%, at least 2%, at least 3%, at least 4%, at least 5%, at least 10%, at least 20%, at least 30%, at least 40% of the length of a polypeptide, such as a polypeptide having an amino acid sequence identified by a specific SEQ ID NO, or having at least 50%, or at least 60%, or at least 70%, or at least 80%, or at least 90% of the length (in amino acids) of the polypeptide.

Within the context of the application a protein is represented by an amino acid sequence and correspondingly a nucleic acid molecule or a polynucleotide represented by a nucleic acid sequence. Identity and similarity between sequences: throughout this application, each time one refers to a specific amino acid sequence SEQ ID NO: (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having Sulphur-containing side chains is cysteine and methionine. Preferred conservative amino acids substitution groups are valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s).

As used herein, a “regulatory sequence” refers to any genetic element that is known to the skilled person to drive or otherwise regulate expression of nucleic acids in a cell. Such sequences include without limitation promoters, transcription terminators, enhancers, repressors, silencers, kozak sequences, polyA sequences, and the like. A regulatory sequence can, for example, be inducible, non-inducible, constitutive, cell-cycle regulated, metabolically regulated, and the like. A regulatory sequence may be a promoter. As used herein, the term “promoter” refers to a nucleic acid fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions.

The term “fusion protein” or fusion polypeptide” are used interchangeably and refer to a polypeptide encoded by a nucleic acid sequence containing a coding sequence from one nucleic acid molecule and the coding sequence from another nucleic acid molecule in which the coding sequences are in the same reading frame such that when the fusion construct is transcribed and translated in a host cell, the protein is produced containing the two proteins. The two molecules can be adjacent in the construct or separated by a linker polypeptide that contains, 1, 2, 3, or more amino acids. The protein product encoded by a fusion construct is referred to as a fusion polypeptide.

The terms “host cell,” “host cell line,” “host cell culture” “genetically modified” or “engineered host” cell as used interchangeably herein, refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein. In some aspects, the host cell is a genetically modified cell. As defined herein in such a cell the nuclear, organellar or extrachromosomal nucleic acid may have been transformed, modified or transduced using recombinant DNA technology to comprise a heterologous nucleic acid molecule, and is used interchangeably with “engineered cell,” “transformed cell,” and “transduced cell.” In one aspect, a genetically modified cell as disclosed herein expresses a protein encoded by a nucleic acid molecule engineered in such manner to contain an insertion of at least one nucleotide, a deletion of at least one nucleotide, and/or a substitution of at least one nucleotide in a sequence encoding at least one ribosomal protein gene. In some aspects, the engineered cell has an insertion of a SNAP/CLIP/AGTanalog-tag into a ribosomal gene sequence. An engineered cell may refer to a cell in isolation or in culture. Engineered cells may be “transduced cells” wherein the cells have been infected with e.g., a modified virus, for example, a retrovirus may be used but other suitable viruses may also be contemplated such as lentiviruses. Non-viral methods may also be used, such as transfections. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient but may also be a stable transfection wherein cells can be selected that have the gene construct integrated in their genome. In some cases, genetic engineering systems such as CRISPR or Argonaute may be utilized to design engineered cells that express a polypeptide described herein.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems. A CRISPR system can be utilized to facilitate insertion of a polynucleotide sequence encoding a membrane protein or a component thereof into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA.

As used here the term “substrate” refers to a benzylguanine (BG) or benzylcytosine (BC) derivatives (comprising a labeling moiety or label) that is capable of specifically and irreversibly bind to a O-6-alkylguanine-DNA alkyltransferase (AGT) derivatives and/or a variant for example a SNAP-tag or a CLIP-tag. In some aspects, the substrate may be synthesized to comprise BG or BC attached to any label that imparts the desired property to the fusion protein, for example spectroscopic probes, fluorescent probes, reporter molecules, affinity tags, molecules generating reactive radicals, cross-linkers, ligands mediating protein-protein interactions or molecules suitable for the immobilization of the fusion protein. Methods of synthesizing these probes are well known in the field. In some aspects, the substrate may be synthesized or may be commercially available.

The term “labeling” as used herein refers to contacting a fusion protein comprising a O-6-alkylguanine-DNA alkyltransferase derivative for example a SNAP-tag or a CLIP-tag with a suitable substrate which comprises a labeling moiety or label.

### II. Compositions

In some aspects, the current disclosure is based on the surprising discovery that ribosomal protein RPL28 can be expressed as a fusion protein with an O-6-alkylguanine-DNA alkyltransferase derivative commercially available as the SNAP-tag® and incorporated to form a fully functional ribosome. Not only does this allow for the visualization of eukaryotic ribosomes in vivo, ex vivo and in vitro, it also enables a myriad of studies that require spatial or temporal differential labeling of populations of ribosomes. In addition, it can be used for example, to determine binding partners for ribosomal protein RPL28. A similar approach is envisaged for other ribosomal protein exposed at the surface of the ribosome fused to O-6-alkylguanine-DNA alkyltransferase (AGT) derivatives, fragments and/or variant like SNAP and CLIP-tag.

In some aspects, the current disclosure encompasses a fusion polypeptide comprising an amino acid sequence corresponding to a O-6-alkylguanine-DNA alkyltransferase analog, variant and/or derivate and a ribosomal protein. O-6-Alkylguanine-DNA alkyltransferase (AGT) is a DNA repair protein that protects from alkylating agents by converting O-6-alkylguanine to guanine forming S-methylcysteine in the AGT protein.

SNAP-tag (SEQ ID. NO. 1) is a 20 kDa mutant of AGT that reacts specifically and rapidly with benzylguanine (BG) derivatives, leading to irreversible covalent labeling of the SNAP-tag with the labeling moiety. SNAP-tag and related derivatives have a number of features that make it ideal for a variety of applications in protein labeling. The rate of the reaction of SNAP-tag with BG derivatives is to a large extent independent of the nature of the synthetic probe attached to BG, permitting the labeling of SNAP fusion proteins with a wide variety of synthetic probes. SNAP-tag has no restrictions with respect to cellular localization and expression host. SNAP-tag substrates are chemically inert towards other proteins, avoiding nonspecific labeling in cellular applications. Many SNAP-tag substrates are cell permeable, permitting labeling of intracellular proteins in live cells.

CLIP-tag (SEQ ID. NO: 2) is a modified version of SNAP-tag. It is also a self-labeling protein derived from human O-6-alkylguanine-DNA-alkyltransferase. Instead of benzylguanine derivatives, CLIP tag is engineered to react with benzylcytosine derivatives. The CLIP-tag was created by engineering the substrate specificity of the SNAP-tag, permitting it to react specifically with O-2-benzylcytosine (BC) derivatives. Since the SNAP- and CLIP-tags specifically react with orthogonal substrates, in some aspects SNAP and CLIP fusion proteins can be labeled simultaneously and specifically with different labels in living cells. One application of the CLIP-tag is dual-labeling of fusion proteins in conjunction with the SNAP-tag.

In some aspects, the current disclosure encompasses a fusion protein comprising a polypeptide comprising a SNAP-tag or CLIP-tag or other O-6-alkylguanine-DNA alkyltransferase analogs, variants and/or a derivates and a ribosomal protein. In some aspects, the fusion protein comprises a polypeptide sequence at least about 70% to about 75%, or about 75% to about 80%, or about 80% to about 85%, or about 85% to about 90%, or about 90% to about 95%, or about 95% to about 100% identical to any one of SEQ ID NOS: 1 or 2.

In some aspects, of the current disclosure, the fusion polypeptide may comprise an amino acid sequence corresponding to any ribosomal protein (or fragments, variants and/or derivatives thereof) that at least in part is exposed on the surface of the ribosome. In some aspects, the ribosomal protein may be a prokaryotic ribosomal protein. In some aspects, the ribosomal protein may be a eukaryotic ribosomal protein. In some aspects, the eukaryotic ribosomal protein is incorporated or can be incorporated into the large subunit of the ribosome. In other aspects, the eukaryotic ribosomal protein is incorporated or can be incorporated into the small subunit of the ribosome. In some aspects, the ribosomal protein sequence corresponds to the corresponding wild-type protein. In other aspects, the ribosomal protein sequence corresponds to a mutant, variant, or fragment with respect to the wild-type protein.

In some exemplary aspects, the ribosomal protein is RPL28. The fusion protein can comprise a polypeptide sequence comprising SEQ ID. NO: 3 or variants, derivatives or fragments thereof. In some aspects, the fusion protein comprises a polypeptide sequence at least about 70% to about 75%, or about 75% to about 80%, or about 80% to about 85%, or about 85% to about 90%, or about 90% to about 95%, or about 95% to about 100% identical to SEQ ID NOS: 3 or a fragment thereof.

In some aspects, the fusion protein may further comprise additional sequences for example linker sequences, degrons, degradation tags, protease cleavage sites, and/or purifications tags.

In some aspects, the current disclosure also encompasses polynucleotides comprising a nucleic acid sequence encoding the fusion proteins disclosed herein. In some aspects, the polynucleotide comprises a nucleic acid sequence at least about 60% identical to SEQ ID NOS: 4 or 5. In some aspects, the nucleic acid sequence is at least about 60% to about 70%, or about 70% to about 75%, or about 75% to about 80%, or about 80% to about 85%, or about 85% to about 90%, or about 90% to about 95%, or about 95% to about 100% identical to any one of SEQ ID NOS: 6.

In some aspects, the polynucleotide further comprises a nucleic acid sequence at least about 60% identical to SEQ ID NOS: 6. In some aspects, the nucleic acid sequence is at least about 60 to about 70%, or about 70% to about 75%, or about 75% to about 80%, or about 80% to about 85%, or about 85% to about 90%, or about 90% to about 95%, or about 95% to about 100% identical to any one of SEQ ID NO: 6.

In some aspects, the current disclosure encompasses a composition comprising a polynucleotide encoding the fusion protein provided herein. In some aspects, the polynucleotide encoding the fusion polypeptide is incorporated into a vector sequence for transduction or transfection into a host cell. In some aspect any suitable vector may be used. In some aspects, the vector is a viral vector. Non-limiting examples of suitable viral vectors include but are not restricted to adenovirus, adeno associated virus (AAV), retrovirus, herpesvirus, lentivirus, poxvirus, or papilloma virus vector. In some aspects, the viral vector is an RNA viral vector. In some aspects, the viral vector is a DNA viral vector. In some aspects, the vector is a DNA or an RNA sequence incorporated into a suitable delivery means. In some aspects, the delivery means could be a lipid nanoparticle.

In some aspects, the polynucleotide is part of a construct used in a CRISPR/Cas system for incorporation into the host genome. In such aspects, the polynucleotide encoding the ribosomal fusion protein further comprises left (LHA) and right homology arms (RHA) that flank the sequence encoding the ribosomal protein. The homology arms have substantial sequence homology to genomic DNA at or near the targeted insertion site. For example, the left homology arm can be a nucleotide sequence homologous with a region located to the left or upstream of the target site or cut site and the right homology arm can be a nucleotide sequence homologous with a region located to the right or downstream of the target site or cut site. The proximal end of each homology arm can be homologous to genomic DNA sequence abutting the cut site. Alternatively, the proximal end of each homology arm can be homologous to genomic DNA sequence located up to about 10, 20, 30, 40, 50, 60, or 70 nucleobases away from the cut site. As such, the polynucleotide encoding the fusion protein can be inserted into the targeted gene locus within about 10, 20, 30, 40, 50, 60, or 70 base pairs of the cut site. The homology arms can range in length from about 50 nucleotides to several of thousands of nucleotides. In some aspects, the homology arms can range in length from about 500 nucleotides to about 1000 nucleotides. In some embodiments, the homology arms are 600 bp, 700 bp, 800 bp, or 900 bp. In some embodiments, the homology arms are 800 bp. In some aspects, the substantial sequence homology between the homology arms and the genomic DNA is at least about 80%, at least about 85%, at least about 90%, at least about 95%, or at least about 99%. In some exemplary aspects, the homology arms have 100% sequence identity with genomic DNA flanking the target site. In some particular aspects, the target site is the gene encoding RPL28. In some aspects, the polynucleotide sequence encoding the fusion polypeptide further comprises a RHA at least about 80% identical to SEQ ID NO: 7. In some aspects, the polynucleotide sequence encoding the fusion polypeptide further comprises LHA at least about 80% identical to SEQ ID NO: 8. In some aspects, the polynucleotide sequence encoding the fusion polypeptide further comprises a sequence at least about 90%, 91, 92%, 93%, 94%, 95%, 96%, 97%, 98%, 99% or 100% identical to SEQ ID NOS: 7 and 8.

In some aspects, the polynucleotide sequence encoding the ribosomal protein fusion polypeptide replaces the polynucleotide sequence encoding the wild-type ribosomal protein on the genome of an engineered host cell. In some aspects, the polynucleotide encoding the ribosomal fusion protein is comprised extra chromosomally or episomally or at a locus different from the endogenous locus for the ribosomal gene in the host cell. In some aspects, the polynucleotide encoding the ribosomal fusion protein is operably linked to an exogenous promoter. In some embodiments, the exogenous promoter can be a constitutive, inducible, temporal-, tissue-, or cell type-specific promoter. In some embodiments, the exogenous promoter is a CAGGS, CMV, EFla, PGK, CAG, or UBC promoter. In some aspects, the polynucleotide sequence further comprises one or more regulatory sequences selected from an enhancer, a leader, a transcription start site (TSS), a linker, 5′ and 3′ untranslated regions (UTRs), an intron, a polyadenylation signal, and a termination region or sequence.

In some aspects, the current disclosure also encompasses a host cell or engineered cell or genetically modified cell comprising one or more polynucleotide encoding the fusion polypeptide disclosed herein. In some aspects, the engineered host cell or a population of host cells comprises a fusion polypeptide comprising an amino acid sequence corresponding to an AGT analog, variant or a derivative (for example a SNAP or CLIP tag) and a ribosomal protein or variant, derivative or fragment thereof. In some aspects, the current disclosure encompasses a engineered host cell or population of host cells comprising one or more fusion polypeptides disclosed herein. In some aspects, the fusion polypeptide is incorporated into the ribosome of the engineered host cell. In some aspects, the fusion polypeptide is not incorporated into the ribosome of the engineered host cell.

In some aspects, the host cell is a prokaryotic cell. In some aspects, the host cell is a eukaryotic cell. In some aspects, the host cell is an animal, plant or yeast cell. In some exemplary aspects, the host cell is a human cell. In some aspects, the host cell is an in vitro cell line or an ex vivo cell. In some aspects, the host cell is present in vivo. In some aspects, the host cell is a somatic cell. In some aspects, the host cell is a differentiated cell. In some aspects, the host cell is a stem cell. In some aspects, the host cell is an embryonic stem cell (ESC), an adult stem cell (ASC), an induced pluripotent stem cell (iPSC), or a hematopoietic stem or progenitor cell (HSPC). In some aspects, the host cell is selected form the group comprising: CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; Hela cells; HT-1080 cells; HCT116 cells; Hu-h7 cells; Huvec cells; Molt 4 cells. In some exemplary aspects, the host cell is selected from a HEK293T, HCT116, iPS or hES cell.

### III. Methods

In some aspects, the current disclosure also encompasses methods of making and using a cell or population of cells comprising a SNAP, CLIP, and/or any AGT analog-tagged-ribosome. In some aspects, such a cell or cell population is engineered to comprise one or more ribosomal proteins tagged with a SNAP/CLIP tag or any suitable variant or derivative thereof. In some aspects, the tagged ribosomal protein may be incorporated into the ribosomes or population of ribosomes in a cell. The tagged ribosomal protein can be used to study for example, the diversity, complexity, dynamics, and regulation of ribosome production and aspects of ribosome function. In some aspects, the tag may be labeled by contacting the cell/ribosome or ribosomal protein with a suitable substrate. In some aspects, a single substrate can be used. In some aspects, multiple substrates can be used to differentially label ribosomes in a spatial or temporal manner. In some aspects, the tag may be used to purify, for example, intact ribosomes, ribosomal subunit, ribosomal proteins, and/or interacting partners. In some aspects, mutants/variants of the tagged ribosomal protein can be incorporated into the ribosomes and functionally assessed. In some particular aspects, the method provided herein allows for the differential labeling of pre-existing and newly produced ribosomes in living cells. This technique allows tracking and quantification of the level old and new ribosomes in living cells and the maturation rate of pre-ribosomes across different human cells. In some aspects, the technique can be used for heterochronic labeling of ribosomes in different cell types.

In some aspects, the method of making cells disclosed herein comprises contacting a host cell with a composition comprising the polynucleotide sequence disclosed herein. Methods for engineering cells to express a desired fusion polypeptide are well known in the art and disclosed herein. Use of recombinant DNA techniques known in the art is envisaged to make constructs provided herein.

In some exemplary aspect the method of making a cell comprising a polynucleotide disclosed herein, comprises delivering to a cell: (a) a first site-directed nuclease targeting a site within or near a gene that encodes a ribosomal protein; and (b) a nucleic acid comprising a nucleotide sequence encoding a CLIP-tagged and/or SNAP-tagged ribosomal protein that is flanked by (i) a nucleotide sequence homologous with a region located left upstream of the target site of (a) and (ii) a nucleotide sequence homologous with a region located right downstream of the target site of (a), wherein the site-directed nuclease cleaves the target site of (a) and the nucleic acid of (b) is inserted at a site that partially overlaps, completely overlaps, or is contained within, the site of (a). In some aspects, the site-directed nuclease is an RNA-guided nuclease. In some aspects, a ribonucleoprotein particle (RNP) containing an RNA-guided nuclease (e.g., a Cas nuclease, such as a Cas9 nuclease) and a gRNA targeting the ribosomal gene to be replaced are delivered to any cell described herein (e.g., HEK293T). A RNP is an RNA-guided nuclease (e.g., Cas9) pre-complexed/complexed with a gRNA. In other aspects, the RNA-guided nuclease and gRNA are delivered separately to cells. In some aspects, the site-directed nuclease targeting a site is introduced into a cell along with a polynucleotide composition comprising a polynucleotide a nucleic acid comprising a nucleotide sequence encoding a CLIP-tagged and/or SNAP-tagged ribosomal protein that is flanked by (i) a nucleotide sequence homologous with a region located left upstream of the target site and (ii) a nucleotide sequence homologous with a region located right downstream of the target site. In some aspects, the composition may comprise a viral vector or any other suitable delivery system provided herein.

In some exemplary aspects, the target site is the ribosomal gene is RPL28. In this aspect the gRNA targets a site near or at RPL28. In some exemplary aspect the guide RNA corresponds to SEQ ID NO 9. In this exemplary aspect the polynucleotide sequence encoding the fusion polypeptide comprises a nucleic acid sequence at least about 60% identical to one or more of SEQ ID NOS: 6, 7 and 8.

In some aspects, a population of cell may be engineered. In some aspect, at least 50% of the engineered cells of a population of cells expresses a detectable level of the protein encoded by the inserted polynucleotide encoding the fusion protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered cells of a population express a detectable level of the fusion protein encoded by the inserted polynucleotide.

In some aspects, the current disclosure encompasses methods of using the engineered host cell expressing the fusion polypeptide. A wide-range of application can be envisaged and some exemplary uses are provided in the examples and detailed description. Depending on the application, a range of substrates for SNAP, CLIP and/or any other AGT analogs can be used. These substrates carry a suitable label based on the application. Non-limiting examples of suitable labels and potential applications are provided here:

### 1) the Label as a Spectroscopic Probe or Reporter Group

In such an aspect the substrate carries a detectable label which can be transferred to the SNAP/CLIP/AGT analog, such as a fluorophore, a chromophore, a magnetic probe, a radioactively labelled molecule or any other spectroscopic probe, and allows the disclosed technique to be used to specifically and covalently attach the detectable label to the fusion protein, either in vivo, ex vivo or in vitro. This allows the detection, visualization and characterization of the fusion protein and complexes for example ribosomes comprising the fusion protein in vivo or in vitro. The term in vivo includes labelling in all compartments of a cell. In an exemplary aspect, it includes labeling on a ribosome.

In this aspect, and by way of non-limiting example, the label may be chosen from the following luminescent compounds or fluorophores: organic fluorophores such as cyanines and derivatives thereof, (in particular those known under the tradenames DY647, Cy5, DY490), fluorescein and derivatives thereof, coumarin and derivatives thereof, rhodamine and derivatives thereof, carbopyronine and derivatives thereof, oxazine and derivatives thereof, AlexaFluors, Crystal Violet and derivatives thereof, perylene bisimides and derivatives thereof, squaraines, BODIPYs, NBD (nitrobenzoxadiazole) and derivatives thereof, DABCYL (4-((4-(dimethylamino)phenyl)azo)benzoic acid) and derivatives thereof; protein fluorophores such as GFP and variants thereof, fluorescent proteins extracted from corals, phycobiliproteins, such as B-phycoerythrin, R-phycoerythrin, C-phycocyanin, allophycocyanins, in particular those known under the name XL665; fluorescent lanthanide complexes such as lanthanide cryptates, lanthanide chelates (in particular the chelates and cryptates of europium, terbium, samarium, dysprosium, neodymium). In some aspects, the label may also be a non-fluorescent energy acceptor and for example may be chosen from the products known under the following tradenames: the products QXL from Anaspec and in particular the products QXL 570, QXL 610, QXL 670 and QXL 680; the products DYQ660 and DYQ661 from Dyomics; the products QSY7, QSY9 and QSY21 from Invitrogen; the products ATTO540Q, ATTO580Q, ATTO621Q from ATTO-TEC. In some aspects, the label is chosen from the products known under the following tradenames: DY647; d2; ATTO647N; ATTO465; fluorescein-5-EX; 5,6-carboxyfluorescein; europium Py-bipy cryptate; terbium DOTA-TATP complex; europium py-bipy-tetraacid cryptate, europium tris-bipy cryptate, terbium cryptate Tb(KR), europium trisbipy-pentaacid cryptate, europium Py-biTATP cryptate.

Suitable applications for using these substrates include but are not restricted to pulse labeling, heterochronic labeling, simultaneous labeling of multiple fusion protein expressing different tags and/or visualization.

For example, a engineered host cell expressing a ribosomal protein comprising a SNAP tag can be contacted sequentially with three different SNAP tag substrates (for example TMR-Star [red fluorescent], non-fluorescent, and Oregon green [green fluorescent]) to separately label pre-existing and newly produced ribosomes. The SNAP tag performs a suicide reaction and cannot react with other substrates after its first labeling, which ensures that red and green labeling mark chronologically distinct ribosome populations. These populations of ribosomes can then be visualized inside the cell or isolated and the differences in composition can be determined.

In another exemplary aspects, two ribosomal proteins can be tagged, one with a SNAP tag and one with a CLIP tag and labeled differently with different substrates. These dual tags can be utilized for differential but simultaneous labeling of populations of ribosomes.

### 2) the Label as a Tag to Detect and Isolate Fusion Protein and Ribosomes

The use of substrates, such as O-6-benzylguanine or O-2-benzylcytosine derivatives, which are labelled with an affinity tag such as biotin allows the present method to be used to transfer an affinity tag to the fusion protein, thereby allowing the fusion protein to be bound by a binding partner of the affinity tag. By way of example, biotin, streptavidin or avidin labeled substrate may be contacted to engineered cells expressing the fusion protein, or to the cell extracts of such cells or to purified fusion proteins, leading to the covalent modification of the fusion protein with the affinity tag. This will then allow the isolation of the fusion protein using the interaction between the affinity tag and its binding partner, e.g. in the case of biotin, immobilized avidin or streptavidin. If the label is linked to the fusion protein via a linker containing a cleavable bond, such as a disulfide bridge, or if the linker is photocleavable, the fusion protein can be released from the affinity tag after its isolation. In some exemplary aspects, SNAP or CLIP-tag-Trap can be used to isolate fusion protein and ribosomes.

### 3) the Label as of Source of Reactive Radicals

Substrates, such as O-6-benzylguanine or O-2-benzylcytosine derivatives can be used to introduce labels into fusion proteins which are capable of generating reactive radicals, such as hydroxyl radicals, upon exposure to an external stimulus. The generated radicals can then inactivate the fusion proteins as well as those proteins that are in close proximity of the fusion protein, allowing the study the role of these proteins. Examples of such labels are tethered metal-chelate complexes that produce hydroxyl radicals upon exposure to H₂O₂ and ascorbate, and chromophores such as malachite green that produce hydroxyl radicals upon laser irradiation. The use of chromophores and lasers to generate hydroxyl radicals is also known in the art as chromophore assisted laser induced inactivation (CALI). CALI is a method that is used to specifically inactivate certain proteins within a cell in a time-controlled and spatially resolved manner and which is based upon the spatial neighborhood of a chromophore and a protein.

In a similar manner, fusion proteins can be labeled with tethered metal-chelates and the fusion protein and those proteins that interact with the fusion protein can be inactivated in a specific manner upon exposure to H₂O₂ and ascorbate. The method can not only be used to study the function of a fusion protein or those that are in close proximity of the fusion protein, but also to identify those proteins that are in close proximity of a fusion protein. Here, proteins which are in proximity of the fusion protein can be identified as such by either detecting fragments of that protein by a specific antibody, by the disappearance of those proteins on a high-resolution 2D-electrophoresis gels or by identification of the cleaved protein fragments via separation and sequencing techniques such as mass spectrometry or protein sequencing by N-terminal degradation.

### 4) the Label as a Ligand Mediating Protein-Protein Interactions

The use of labeled substrates, such as O-6-benzylguanine or O-2-benzylcytosine derivatives can be used to transfer a ligand to the protein fusion. This allows binding partners of the ligand, such as proteins, to bind to the fusion protein. For example, where the label is a ligand which is capable of binding to a binding partner, contacting such a substrate with a fusion protein will lead to specific attachment of the ligand to the fusion protein. If the ligand binds to another protein Y and the dimerization of the protein Y with the labelled fusion protein leads to a biological function or a measurable signal, the biological function or the measured signal depends on the addition of the substrate carrying the label. In some aspect the label may be an aptamer.

5) the Label as a Library of Small Molecules to Identify Molecules that Bind to the Ribosomal Protein

The use of O-6-benzylguanine or related AGT substrates that carry a label and where the label is a library of chemical molecules: the goal would be to identify small molecules that bind to a ribosomal protein under in vivo conditions.

6) the Label as a Cross-Linker to Detect Proteins that Interact with the Fusion Protein

The use of O-6-benzylguanine derivatives or related AGT substrates which carry a label and where the label is a molecule that can cross-link to other proteins. Examples of such cross-linkers are molecules containing functional groups such as maleimides, active esters or azides and others known to those proficient in the art. Contacting such substrates with fusion proteins that interact with other proteins (in vivo, ex vivo or in vitro) can lead to the covalent and specific cross-linking of the fusion protein with its interacting protein via the label. This allows the identification of the protein interacting with the fusion protein.

In some aspect, the substrate may be commercially available for example BG-GLA-NHS, BG-Maleimide, BG-NH2, BG-PEG-NH2, SNAP-Biotin®, SNAP-Cell® 505-Star, SNAP-Cell® Block, SNAP-Cell® Oregon Green®, SNAP-Cell® TMR-Star, SNAP-Cell® 430, SNAP-Cell® 647-SIR, SNAP-Surface® 488, SNAP-Surface® 549, SNAP-Surface® 594, SNAP-Surface® 649, SNAP-Surface® Alexa Fluor® 488, SNAP-Surface® Alexa Fluor® 546, SNAP-Surface® Alexa Fluor® 647, SNAP-SurfaceR Block, BG-NH2, CLIP-Biotin®, CLIP-Cell® 505-Star, CLIP-Cell® Block, CLIP-Cell® TMR-Star, CLIP-Surface® 488, CLIP-Surface® 547, CLIP-Surface® 547, CLIP-Surface® 647.

### IV. Kits

In some aspects, the compositions and methods provided herein can also be provided in the form of kits with instructions for use. In some aspects, the kits comprise at least a composition comprising a polynucleotide encoding fusion polypeptide provided herein, and optionally suitable substrates, reagents, buffers, diluents, cells, standards, containers and instructions for use. In some aspects, the kits may comprise at least a cell comprising the polynucleotide or polypeptide disclosed herein and optionally suitable substrates, reagents, buffers, diluents, cells, standards, containers and instructions for use.

In some aspects, the article of manufacture or kit comprises a container and a label or package insert on or associated with the container. Suitable containers include, for example, microfuge tubes, bottles, vials, assay plates, strips, matrices etc. The containers may be formed from a variety of materials such as glass, plastic, paper etc. The kit may further include other materials desirable from a commercial and user standpoint, including other buffers, diluents, filters, needles, and syringes.

A “package insert” is used to refer to instructions customarily included in commercial packages of products, that contain information about usage etc.

Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

TABLE 1
Sequences

| SEQ | | |
|---|---|---|
| ID | | |
| NOS | NAME | SEQUENCE |
| 1 | SNAP tag | DKDCEMKRTTLDSPLGKLELSGCEQGLHEIKLLGKGTSAADAVEVP |
| | | APAAVLGGPEPLMQATAWLNAYFHQPEAIEEFPVPALHHPVFQQE |
| | | SFTRQVLWKLLKVVKFGEVISYQQLAALAGNPAATAAVKTALSGNP |
| | | VPILIPCHRVVSSSGAVGGYEGGLAVKEWLLAHEGHRLGKPGLG |
| 2 | CLIP tag | DKDCEMKRTTLDSPLGKLELSGCEQGLHRIIFLGKGTSAADAVEVP |
| | | APAAVLGGPEPLIQATAWLNAYFHQPEAIEEFPVPALHHPVFQQES |
| | | FTRQVLWKLLKVVKFGEVISESHLAALVGNPAATAAVNTALDGNPV |
| | | PILIPCHRVVQGDSDVGPYLGGLAVKEWLLAHEGHRLGKPGLG |
| 3 | RPL28 | MSAHLQWMVVRNCSSFLIKRNKQTYSTEPNNLKARNSFRYNGLIH |
| | | RKTVGVEPAADGKGVVVVIKRRSGQRKPATSYVRTTINKNARATLS |
| | | SIRHMIRKNKYRPDLRMAAIRRASAILRSQKPVMVKRKRTRPTKSS |
| 4 | SNAP tag | GACAAAGATTGCGAAATGAAACGTACCACCCTGGATAGCCCTCT |
| | DNA | GGGCAAGCTGGAACTGTCTGGGTGCGAACAGGGCCTGCACGAG |
| | | ATCAAGCTGCTGGGCAAAGGAACATCTGCCGCCGACGCCGTGG |
| | | AAGTGCCTGCCCCAGCCGCCGTGCTGGGCGGACCAGAGCCACT |
| | | GATGCAGGCCACCGCCTGGCTCAACGCCTACTTTCACCAGCCT |
| | | GAGGCCATCGAGGAGTTCCCTGTGCCAGCCCTGCACCACCCAG |
| | | TGTTCCAGCAGGAGAGCTTTACCCGCCAGGTGCTGTGGAAACT |
| | | GCTGAAAGTGGTGAAGTTCGGAGAGGTCATCAGCTACCAGCAG |
| | | CTGGCCGCCCTGGCCGGCAATCCCGCCGCCACCGCCGCCGTG |
| | | AAAACCGCCCTGAGCGGAAATCCCGTGCCCATTCTGATCCCCTG |
| | | CCACCGGGTGGTGTCTAGCTCTGGCGCCGTGGGGGGCTACGA |
| | | GGGCGGGCTCGCCGTGAAAGAGTGGCTGCTGGCCCACGAGGG |
| | | CCACAGACTGGGCAAGCCTGGGCTGGGT |
| 5 | CLIP tag | GACAAAGACTGCGAAATGAAGCGCACCACCCTGGATAGCCCTCT |
| | DNA | GGGCAAGCTGGAACTGTCTGGGTGCGAACAGGGCCTGCACCGT |
| | | ATCATCTTCCTGGGCAAAGGAACATCTGCCGCCGACGCCGTGG |
| | | AAGTGCCTGCCCCAGCCGCCGTGCTGGGCGGACCAGAGCCACT |
| | | GATCCAGGCCACCGCCTGGCTCAACGCCTACTTTCACCAGCCT |
| | | GAGGCCATCGAGGAGTTCCCTGTGCCAGCCCTGCACCACCCAG |
| | | TGTTCCAGCAGGAGAGCTTTACCCGCCAGGTGCTGTGGAAACT |
| | | GCTGAAAGTGGTGAAGTTCGGAGAGGTCATCAGCGAGAGCCAC |
| | | CTGGCCGCCCTGGTGGGCAATCCCGCCGCCACCGCCGCCGTG |
| | | AACACCGCCCTGGACGGAAATCCCGTGCCCATTCTGATCCCCTG |
| | | CCACCGGGTGGTGCAGGGCGACAGCGACGTGGGGCCCTACCT |
| | | GGGCGGGCTCGCCGTGAAAGAGTGGCTGCTGGCCCACGAGGG |
| | | CCACAGACTGGGCAAGCCTGGGCTGGGT |
| 6 | RPL28 gene | CTCTTTCCGTCTCAGGTCGCCGCTGCGAAGGGAGGTGAGCGTT |
| | sequence | CGTCTTCCTCGCGTGGTCGCCATCTCCGGCCCGCCGCGCACTC |
| | | CCTCTCAGTCCTCTGCCTGCCTTCTCCCCTTGCGCCTCAAGGGT |
| | | CATTGGCGGACCCCAACCCCTCCCCGTGAAGTCGTTGTGAATTT |
| | | TCAAGTTATTTTCCCCTCACTCTCATTCGCCGCAGCTAGTCTCGG |
| | | CTGCCTGAATTCGGTCCCGCCGCCTTAGGTCACTCAGTCCCGC |
| | | CTGACAAGAGTTCTAGGTCGCTGTCTGCCCTCAGGGGTCTCCCA |
| | | TTCACCCAAGTTCCCAGTCGCTCTCTTCCTCTGCTGTCCGAGCG |
| | | TGGCCCGTGGCCTAACGCTGCTTTAGTTCTCTGTGTCTGACGCT |
| | | TTCCCTGTGCCCGTTTCCCCGCAGCCGCCGCCATGTCTGCGCA |
| | | TCTGCAATGGATGGTCGTGCGGAACTGCTCCAGTTTCCTGATCA |
| | | AGAGGAATAAGCAGACCTACAGCACTGTAAGTGGGGCCCGGAT |
| | | GCGTGGCTCCTGCGGGAGGAGGGTCCTGAGTCTCTTGACTCTG |
| | | GGTCAGAGGGCGGGACCTTCGCATGTCTCCGGGTCCCTTATTC |
| | | ACGATGCCTTGTGCCGCCTCCTTCCCAGGAGCCCAATAACTTGA |
| | | AGGCCCGCAATTCCTTCCGCTACAACGGACTGATTCACCGCAAG |
| | | ACTGTGGGCGTGGAGCCGGCAGCCGACGGCAAAGGTGTCGTG |
| | | GTGGTCATTAAGCGGAGATCCGGTGAGTTTTGTCTGGTTTGGGC |
| | | CAGAGAGCGGCCCCTTTCCCGGGTCTGGGAGCTGTGATTTTTTA |
| | | CTGTCAGGCAGGAAGAGCGGTAACTGCCATCGCGGCGGGCATC |
| | | CCTGGCGCCAGGGTGTTGGTCTGGGTACCGGCTTCCCTCTCGG |
| | | CCGACTTGTCAGCTCTGTGAGCCGCGCGCGTCTGAGCCCGTGT |
| | | CCTCACCTGTAAAGTGGAGAAATGAAAAAGGACCTGAACTTCCT |
| | | CGGTGGTTGTTGAGAGTTAAGGCACGGGGTTGATGTTTTCAGAT |
| | | GAAATTCTCAAAGCAAGTCAGGGTGGGGATGGATGGTTTCATCC |
| | | CACAGGTGGGAAGATTGAGGCACAACAGGGGAGGGGCCCTCG |
| | | CTACCACACTTAGGAGGGGACAGGCTGGGTTGGTTGCAGCCAT |
| | | TTGATTCCCATTTTGCCTGTGTAGGTTAGAGAAGAGGTCTTGGG |
| | | GACCCCACTCCATACATTGTGCTGTCAGGTGCAGGCCTTTTTGG |
| | | GGATTCCCTGAATGTTCGTGTGAGTCGGGGGTCTCTAATGGAGG |
| | | AGTCCAGCTTCACATGTGTTCTTGACAGGGCTGTATTTTCTTTTT |
| | | AGAGTGAGTTTTTCTCAGGTCCTTGATTGGAACTGCCTCAGAGC |
| | | CAAGGGTCCTTTTACTCAGTGGCAGCAACAAACGCAGTCTGTTG |
| | | GCTAGTGATCCTCCTGTCTCAGGGACACGTAGTCCAGGGAGCA |
| | | GCCAATTGCTTGGCACTTGGGGACCCCGTTCTGGGGAGTCCTG |
| | | AAAGCTTTCACCTCTTGGATTGCCGAATACATGGGTGGCCCTTC |
| | | CTAGACTAAGGGACTGGCCTGAGTGAGGCTGGGCCTCTCAGCC |
| | | AAGCTGATGTTGAACCACTGCTGTGGGGATGGGCCTGGGGTTC |
| | | CTGGGAAGCTGTTCATACCCATTGCCAGGAGCGTGGGCTCTGG |
| | | CTGGACCTGGATCAGATCCTAACTGAAGCGGCAGCTTTCTGGCA |
| | | TGAGAAAGGAGTGTTTTCATGGTGGACAGAATTGGGCTATGAGT |
| | | GTGGCAGAAGCTGTGTCCTCAGTACTCCGTGATGACGAGTGAG |
| | | CCTCTGTGAAATGGACAGGTGGGAAAACAGCTACCTGCTGGCCT |
| | | GCCCAGGCACCCGCCACGGGCCCACGCTGCTCAGCTTCTCAAT |
| | | GTGAGACTGTCCACACCTGCGAGGTGTGCTAAAGGTGCAGGTT |
| | | AGGTGGACTGACCCCAGGACCTCCCTGACCCCCAACCAGGCCA |
| | | GCGGAAGCCTGCCACCTCCTATGTGCGGACCACCATCAACAAG |
| | | AATGCTCGCGCCACGCTCAGCAGCATCAGACACATGATCCGCAA |
| | | GAACAAGTACCGCCCCGACCTGCGCATGGTGAGCTGGGGTTTG |
| | | GGGATCAGGCTTGGGGAGACTGGCCAGTGCTGTGGGGAAGGG |
| | | CCTCCCACTACTGGTTGCAATATGGGCTGGAGAGGGATGGATTC |
| | | TTGCTTTCAGCCTACTCCCCACACCCAGCATTGGCCTAGGGGGC |
| | | GGCTTGTGGAGTGTATGGGCTGAGCCTTGCTCTGCTCCCCCGC |
| | | CCCCAGGCAGCCATCCGCAGGGCCAGCGCCATCCTGCGCAGC |
| | | CAGAAGCCTGTGATGGTGAAGAGGAAGCGGACCCGCCCCACCA |
| | | AGAGCTCCTGAGCCCCCTGCCCCCAGAGCAATAAAGTCAGCTG |
| | | GCTTTCTCACCTGCCTCGACTGGGCCTCCCTTTTTGAAACGCTC |
| | | TGGGGAGCTCTGGCCCTGTGTGTTGTCATTCAGGCCATGTCATC |
| | | AAAACTCTGCATGTCACCTTGTCCATCTGGAGGTGATGTCAATG |
| | | GCTGGCCATGCAGGAGGGGTGGGGTAGCTGCCTTGTCCCTGGT |
| | | GAGGGCAAGGGTCACTGTCTTCACAGAAAAAGTTTGCTGACTTG |
| | | TGATTGAGACCTACTGTCCCATTGTGAGGTGGCCTGAAGAATCC |
| | | CAGCTGGGGCAGTGGCTTCCATTCAGAAGAAGAAAGGCCTTTTC |
| | | TAGCCCAGAAGGGTGCAGGCTGAGGGCTGGGCCCTGGGCCCT |
| | | GGTGCTGTAGCACGGTTTGGGGACTTGGGGTGTTCCCAAGACC |
| | | TGGGGGACGACAGACATCACGGGAGGAAGATGAGATGACTTTT |
| | | GCATCCAGGGAGTGGGTGCAGCCACATTTGGAGGGGATGGGCT |
| | | TTACTTGATGCAACCTCATCTCTGAGATGGGCAACTTGGTGGGT |
| | | GGTGGCTTATAACTGTAAGGGAGATGGCAGCCCCAGGGTACAG |
| | | CCAGCAGGCATTGAGCAGCCTTAGCATTGTCCCCCTACTCCCGT |
| | | CCTCCAGGTGTCCCCATCCCTCCCCTGTCTCTTTGAGCTGGCTC |
| | | TTGTCACTTAGGTCTCATCTCAGTGGCCGCTCCTGGGCCACCCT |
| | | GTCACCCAAGCTTTCCTGATTGCCCAGCCCTCTTGTTTCCTTTGG |
| | | CCTGTTTGCTCCCTAGTGTTTATTACAGCTTGTGAGGCCAGGAG |
| | | TTTGAGACCATCCTAGGCAACATAATGAGACACCGTCTCTAAAAT |
| | | AAAATTAGCTGGGTGTGGTGGTGCACCGCCTGTGGTCCCAGCT |
| | | CCTCAGAGGTTGAGTAGAGGCTGAGGTGAGCGGAGCACTTGAG |
| | | CCAAGAGTATGAGGCTGCAGTGAGCCCATGAGCCCCACCACTA |
| | | CACTCCAGCCTGGAAGACACCATGACACACAGTGAGGCCTGGA |
| | | TGGGGAAAGAGTCCTGCTGTTGATCCTCACATGTTTCCTGGGCA |
| | | CCTAACTCTGTCAGCCACTGCCAGGGACCAAGGATCCAGCATCC |
| | | ATGGCACCCCTGGTTCCTGCCATCCTGGGGTACCCGATTCAAAG |
| | | AAGGACTCTGCTCCCTGTCTGAGACCACCCCCGGCTCTGACTGA |
| | | GAGTAAGGGGACTGTCAGGGCCTCGACTTGCCATTGGTTGGGG |
| | | TCGTACGGGGCTGGGAGCCCTGCGTTTTGAGGCAGACCACTGC |
| | | CCTTCCGACCTCAGTCCTGTCTGCTCCAGTCTTGCCCAGCTCGA |
| | | AGGAGAGCAGATCTGACCACTTGCCAGCCCCTGTCTGCTGTGAA |
| | | TTACCATTTCCTTTGTCCTTCCCTTAGTTGGGTCTATTAGCTCAG |
| | | ATTGAGAGGTGTTGCCTTAAAACTGAGTTGGGTGACTTGGTACC |
| | | TGCTCAGGACCCCCCGCACTGTCCCAATCCCACTCAGGCCCAC |
| | | CTCCAGCTGGCCTCACTCCGCTGGTGACTTCGTACCTGCTCAGG |
| | | AGCCCCCACTGTCCCAGTCCCACTCAGGCCCATCTCTGGCTGG |
| | | CCTCACTGCGCTGGGACTCCGCCTTCATAAGGAGAGCTCACTG |
| | | CTCACGTTAGTAGATGGCCCCTTCTCGTGAGGCCTCTCCCCTGG |
| | | CACCTGCTTCAGTTGTCCTCCACAGCACTGATTTGCAGCCCACA |
| | | AGCTGGCAGGTTTATCTGTCTCATGTTTGTCTTGTGCTGGTGGG |
| | | CAAGGGGTTTGTCTAGCACACCAGCATATAATGAGATGCTTGAT |
| | | GAATGGTGCATATTGAATGTATAAAGCCCACCGGTCCTGAGAGT |
| | | TTGCTCACTGGAGACTTTCTGGAGATGGAGTCTCGCTCTGTTGC |
| | | CCAGGCTGGCGAGTGCAATGGCGCGATCTTGGCTCACTGCAGC |
| | | CTCCACCTCCTGGGTTCAAGCGATTCTCCTGCCTCAGCCTCCCG |
| | | AGTAGCTGGGATTACAGGTGGGTGTCACCACACCCAGCTCAGTA |
| | | TTGTATTTTTAGCAGAGATGGGGTTTCACCATTTTGCCCAGGCTG |
| | | GTTTGGAACTCCTGACTTCAAATTACCCACCTGCCTCAGCCTCC |
| | | CAAAGTGCTGGCATTACAGGCGCTCGAGGCTTTCTGATGTGGCT |
| | | GCTGCTGCTCAGAAGGCCTTGTCCTTAACCACCTCCTTGCCTGC |
| | | CCTGGAGGCTTGTGCCTCTAGGCCCCACCCCCTGTGGAGTCCT |
| | | GCTGGCTTTCTCCATCCCTATCTGAATCCTCCCTGCTGTGTGGC |
| | | CTCCCCTGGTCTCATCCGTAACACAGCCCAGCTTAGTGGGCCTC |
| | | TGTTCCTGCGGGTGGCCAGCCTGTCTGTGTGGCTGGGCTGGGG |
| | | AGGCCACGTCTGGTATCTGAATGCTATCGGTGGGTTGGGGTGG |
| | | AGGAACCAGGAGAGGGCTGGAGGGAGGGAGATGGTCTCAGCC |
| | | CCACAGAGTTTGGAGTCCTCAGTGTGCTGAGCAAACGTGGAGA |
| | | CACCATTTCCCTCCTCTAGACCTCATCTTGGAGAGAGAGATGTT |
| | | GGATGGGGCCATCTATTCCAGCTTTATTCACACAAATCATGTCTG |
| | | TTGGCCTGGAAATTGGAAAACCAGTTAAACCAAAAACATGATATT |
| | | AAGAAAACAGGCAGGCTCACCATAGTAAAAATGCTGAAAGCCAA |
| | | AGACAAAATTGGGAGAACAAAAGAAAAGCGTCTTGTCACATACA |
| | | GAAGGTCCCTGATAAAGTTAGTAGCTGCCCTCATCAGAAACCAG |
| | | GCCCAGGCAGTGGGGACACATCCAGAGTGCTGAAAGAACCTCC |
| | | CCCAGGTCATCCTATCCCCAAGAGTGATGCCCGGCAGCATTCCC |
| | | AGCTCAGGGCTAATGGTTCACGGAAGCCAGGAATCAAACTGCCT |
| | | GGGTTCCAGTCCCAGCTCTGCCAGTTATGCCCAGCTGTGGGGA |
| | | CTTGGGCAGCTCGTTTAGTAGCACCGTGCCTCAGTTTCCCATAT |
| | | GTAAAAGGCCATTTTGAGTGCCTTTCACAGCCCTGCATAAGGCA |
| | | GGTGTCTCAGTGTTCACTGCTGTCTCTCCAGCTCTTAGTCCAGT |
| | | AGCTGCATGGTGAGTGAGCGTAGGGCGCACCCTGGAAGGCTGC |
| | | CAAGCCCAAAGTTGTGCAGAGCGCTGGGGACTCCAGACTCCCC |
| | | ACAGCAGCAGAGACTCGGGACTGAGGCATCCTCTGTTCACAGG |
| | | ACATGCTGGCATCTACTGGGTCAGGGCTCTGCTGCTCGGTGGC |
| | | TGTGCAACCTTGGGCAAGTTCCTCAACCTCTCTGTGTCTTCGTA |
| | | CCCTCATCTGTAACATGCGTGTCGATAGACCCTACTACTCAGGG |
| | | TTGATGAGAAGATTAAATGTGCAAAACCTGCTTGACTGTGCCCA |
| | | CAAATCCTGATTGTAGGAATAAATTAATGACTTTTTATAAATATTT |
| | | TGATCAGATGGACTCATGATCACAGATGTCTTCACATGCCTATGA |
| | | CTAATTTGTACACAAACTAATGCTCGTGTTTCCCAAGCACCTGGA |
| | | AGACATGCCAGATCCATGTGCAGTAATGCCTGGTGGCTCCAGGT |
| | | CTGCCCCGCCGTCCTGTGGGGCTGTGAGCTTTCCCAGCCTCCT |
| | | GCCCGTGTTTGTGAATATCATTCTGTCCTCAGCTGCATTTCCAGC |
| | | CCAGGCTGTTTGGCGCTGCCCAGGAATGGTATCAATTCCCCTGT |
| | | TTCTCTTGTAGCCAGTTACTAGAATAAAATCATCTACTTTAAAA |
| 7 | LHA-RPL28 | GGCTGGGCCTCTCAGCCAAGCTGATGTTGAACCACTGCTGTGG |
| | | GGATGGGCCTGGGGTTCCTGGGAAGCTGTTCATACCCATTGCC |
| | | AGGAGCGTGGGCTCTGGCTGGACCTGGATCAGATCCTAACTGA |
| | | AGCGGCAGCTTTCTGGCATGAGAAAGGAGTGTTTTCATGGTGGA |
| | | CAGAATTGGGCTATGAGTGTGGCAGAAGCTGTGTCCTCAGTACT |
| | | CCGTGATGACGAGTGAGCCTCTGTGAAATGGACAGGTGGGAAA |
| | | ACAGCTACCTGCTGGCCTGCCCAGGCACCCGCCACGGGCCCAC |
| | | GCTGCTCAGCTTCTCAATGTGAGACTGTCCACACCTGCGAGGTG |
| | | TGCTAAAGGTGCAGGTTAGGTGGACTGACCCCAGGACCTCCCT |
| | | GACCCCCAACCAGGCCAGCGGAAGCCTGCCACCTCCTATGTGC |
| | | GGACCACCATCAACAAGAATGCTCGCGCCACGCTCAGCAGCAT |
| | | CAGACACATGATCCGCAAGAACAAGTACCGCCCCGACCTGCGC |
| | | ATGGTGAGCTGGGGTTTGGGGATCAGGCTTGGGGAGACTGGCC |
| | | AGTGCTGTGGGGAAGGGCCTCCCACTACTGGTTGCAATATGGG |
| | | CTGGAGAGGGATGGATTCTTGCTTTCAGCCTACTCCCCACACCC |
| | | AGCATTGGCCTAGGGGGCGGCTTGTGGAGTGTATGGGCGCCTT |
| | | GCTCTGCTCCCCCGCCCCCAGGCAGCCATCCGCAGGGCCAGC |
| | | GCCATCCTGCGCAGCCAGAAGCCTGTGATGGTGAAGAGGAAGC |
| | | GGACCCGCCCCACCAAGAGCTCC |
| 8 | RHA RPL28 | GCCTCCCTTTTTGAAACGCTCTGGGGAGCTCTGGCCCTGTGTGT |
| | | TGTCATTCAGGCCATGTCATCAAAACTCTGCATGTCACCTTGTCC |
| | | ATCTGGAGGTGATGTCAATGGCTGGCCATGCAGGAGGGGTGGG |
| | | GTAGCTGCCTTGTCCCTGGTGAGGGCAAGGGTCACTGTCTTCAC |
| | | AGAAAAAGTTTGCTGACTTGTGATTGAGACCTACTGTCCCATTGT |
| | | GAGGTGGCCTGAAGAATCCCAGCTGGGGCAGTGGCTTCCATTC |
| | | AGAAGAAGAAAGGCCTTTTCTAGCCCAGAAGGGTGCAGGCTGA |
| | | GGGCTGGGCCCTGGGCCCTGGTGCTGTAGCACGGTTTGGGGA |
| | | CTTGGGGTGTTCCCAAGACCTGGGGGACGACAGACATCACGGG |
| | | AGGAAGATGAGATGACTTTTGCATCCAGGGAGTGGGTGCAGCC |
| | | ACATTTGGAGGGGATGGGCTTTACTTGATGCAACCTCATCTCTG |
| | | AGATGGGCAACTTGGTGGGTGGTGGCTTATAACTGTAAGGGAG |
| | | ATGGCAGCCCCAGGGTACAGCCAGCAGGCATTGAGCAGCCTTA |
| | | GCATTGTCCCCCTACTCCCGTCCTCCAGGTGTCCCCATCCCTCC |
| | | CCTGTCTCTTTGAGCTGGCTCTTGTCACTTAGGTCTCATCTCAGT |
| | | GGCCGCTCCTGGGCCACCCTGTCACCCAAGCTTTCCTGATTGC |
| | | CCAGCCCTCTTGTTTCCTTTGGCCTGTTTGCTCCCTAGTGTTTAT |
| | | TACAGCTTGTGAGGCCAGGAGTTTGAGACCATCCTAGGCAACAT |
| | | AATGAGACACCGTC |
| 9 | gRNA | CTTTCTCACCTGCCTCGAC |
| | targeting | |
| | RPL28 | |

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

In some aspects, the current disclosure encompasses methods for in-depth study of eukaryotic ribosomes using novel tagging techniques. The method developed herein comprises a “Ribo-SNAP” technique that allows for the differential labeling of pre-existing and newly produced ribosomes in living cells.

Exemplary use of this technique for tracking and quantification of the levels of old and new ribosomes, and the maturation rate of pre-ribosomes across different human cells is presented. The examples presented here exemplify how the technology was crucial in revealing unexpected variability in ribosome assembly rates across different cell types. Using Ribo-SNAP labeling in a genome-scale CRISPR/Cas9 knock out screen, C1ORF109, SPATA5, SPATA5L1 and CINP were identified, which together promote the recycling of RSL24D1 from cytoplasmic pre-60S ribosomal subunits back to the nucleolus, a step that is carried out by a single protein in yeast. Allelic variants of SPATA5, SPATA5L1, and CINP have been linked with microcephaly, intellectual disability, hearing loss, and seizures in human patients. Two allelic variants in SPATA5 compromise ribosome biogenesis, implicating defects in ribosome production and mRNA translation in these disorders.

The development and some exemplary methods for using the disclosed compositions and methods are presented herein.

### Example 1: Materials and Method

### Cell Culture

HEK293T and LentiX-HEK293T cells were grown in Dulbecco's modified Eagle's medium (DMEM) (ATCC) supplemented with 10% fetal bovine serum (ATCC) and 1% Penicillin-Streptomycin (Gibco) and maintained in a 5% CO₂ incubator at 37 °C. HCT116 cells were grown in McCoy's 5A (Modified) Medium (Gibco) supplemented with 10% fetal bovine serum (ATCC) and 1% Penicillin-Streptomycin (Gibco) and maintained in a 5% CO2 incubator at 37 °C. H9 hESCs were cultured in mTeSR Plus media (STEMCELL) on plates coated with Matrigel (Corning) at 37 °C with 5% CO2.

Guide RNA, Probes and Homology Arms Used in this Study

Table 2 provides a list of exemplary guide RNAs optimized to target the indicated genes.

TABLE 2
Exemplary guide RNAs

| | | |
|---|---|---|
| | sgRNA | |
| SEQ ID NOS: | name | Sequence |

gRNA

| | | |
|---|---|---|
| 10 | FBL | ACCAACGATGTCAGAGACAT |
| 11 | TSR2 | CCTGTGTTTACAGATCGCTG |
| 12 | POLR3K | GGGTGATGTTGTGCACGTAG |
| 13 | RPP21 | GTGAAGAGGACTCTCTGTCG |
| 14 | CARS | GCTTCGATGGACATTCACGG |
| 15 | EEF2 | AGACACGCTTCACTGATACC |
| 16 | KIAA1429 | ATGTTAACGGAACTCCTGAG |
| 17 | C1ORF109 | CATCGTCCTGGACAAGCTAG |
| 18 | SPATA5 | ATAAAGTAACACTCCTCTAG |
| 19 | CINP | TCTTACCAACCCATCCAAGG |
| 20 | SPATA5L1 | AAGATCTCGCTACCCGACGG |

Probes

| | | |
|---|---|---|
| 21 | 5′-ETS | /5Biosg/CGGAGGCCCAACCTCTCCGACGACAGGT |
| | | CGCCAGAGGACAGCGTGTCAGC |
| 22 | ITS1 | /5Biosg/CCTCGCCCTCCGGGCTCCGTTAATGATC |
| 23 | ITS2 | /5Biosg/CTGCGAGGGAACCCCCAGCCGCGCA |

### Generation of RPL28-Snap Knock-In Cell Lines

A guide RNA (sgRNA) targeting the C-terminal region of human RPL28 gene was used (see Table 1). The guide sequence for RPL28 (SEQ ID. NO: 9) was assembled into pSpCas9 (BB)-2A-GFP (PX458). Donor vectors were constructed by assembling a snap-tag coding sequence with upstream and downstream homology arms into pUC19 by Gibson assembly (LHA: SEQ ID NO: 7 and RHA: SEQ ID NO: 8). HEK293T and HCT116 cells were transfected with sgRNA and donor vectors (1:3 by molarity) by Lipofectamine 3000 (Thermofisher). 24 hours after transfection, cells were sorted and GFP positive cells were collected. After 7 days' culture, cells were treated with 3 μM SNAP-Cell TMR-Star (NEB S9105S) for 30 min, followed by wash. Cells were sorted and the TMR positive cells were collected into 96-well plates with 1 cell/well. 14 days after the single cell sorting, the colonies were screened for knock-in by genotyping and western blot with antibodies for RPL28 (Abcam 138125). For H9 hESCs, linearized RPL28-SnapTag double stranded oligonucleotide (dsODN) donor was produced by PCR amplification from donor vector. H9 hESCs were electroporated with a NEPA21 Type II Super Electroporator (Bulldog-Bio) using the manufacturer's recommended parameters. After electroporation, cells were collected in 2 mL of mTeSR Plus media supplemented with ROCK inhibitor (Y-27632). After 48 h, EGFP+ cells were sorted and plated at a clonal density (1×10³ cells/mL). Resulting colonies were manually isolated and expanded for genotyping and western blot analysis.

### Cell Proliferation Assay

Wild type HEK293T cells and HEK293T-RPL28-SNAP cells were dislodged, counted and seeded into 6 well plates at 2.0×10⁵ cells/well. Cells were counted 24, 48, 72 h after seeding by Countess Automated Cell Counter (Thermo Scientific) to generate the growth curve.

Random Monolayer Differentiation of RPL28-SnapTag H9 hESCs

RPL28-SnapTag H9 hESCs were dissociated into single cells using Versene (Gibco) and plated at a density of 1.0×10⁵ cells/mL in mTeSR Plus media supplemented with ROCK inhibitor (Y-27632) on dishes coated with Matrigel. After 48 h, the media was replaced with DMEM (SIGMA) supplemented with 10% fetal bovine serum (FBS) and refreshed every other day. After an additional 12 days of culture, the cells were dissociated using TrypLE Express (Gibco) and plated onto Matrigel-coated glass coverslips for a 48 h growth before Ribo-SNAP labeling.

### Embryoid Body Formation

Embryoid bodies (EBs) were generated using AggreWell-800 plates (Stem Cell Technologies) per manufacturer's instructions in mTeSR Plus media with ROCK inhibitor (Y-27632). After 48 h, media was removed and formed EBs were transferred to an ultra-low attachment 24-well plate (Corning) in 2 mL of DMEM supplemented with 10% FBS. After 1 week, EBs were transferred to Matrigel-coated glass coverslips and cultured for an additional week in DMEM media supplemented with 10% FBS, before Ribo-SNAP labeling.

### Ribo-SNAP Labeling

Cells were incubated with 3 μM snap-cell TMR-STAR (NEB) in complete medium for 30 min at 37 °C to label pre-existing ribosomes. After labeling, cells were washed 3 times with complete medium and incubated with complete medium for 30 min to remove unreacted snap substrate. Cells were then grown in complete medium for 24 h. At the time of blocking, cells were incubated with 20 UM snap-cell Block (NEB) in complete medium for 30 min at 37 °C to block unlabeled snap-tag. After blocking, cells were washed 2 times with complete medium and were grown in complete medium until Oregon green labeling. At the time of new ribosome labeling, cells were incubated with 5 UM snap-cell Oregon Green (NEB) in complete medium for 30 min at 37 °C to label newly synthesized ribosomes. After labeling, cells were washed 3 times with complete medium and incubated with complete medium for 30 min to remove unreacted snap substrate. The chase starts at the time of the final medium change.

### Polysome Profiling and Fractionation

Cells were grown to ~80% confluency at the time of assay. Cells were washed once with DPBS and then lysed on cold chamber in lysis buffer (20 mM Tris-Cl pH 7.5, 5 mM MgCl2, 150 mM NaCl, 1 mM DTT, 100 μg/mL cycloheximide (CHX, sigma), 1×complete Protease Inhibitor Cocktail (Roche), 0.1% NP-40). For 100 mm dish, 500 μL lysis buffer was used. The lysate was collected into 1.5 mL tube and stay on ice for 15 min. Lysate was then centrifuged at 21130 g at 4 °C for 10 min to remove nuclei, mitochondria, and debris. RNA concentration of lysates was quantified by nanodrop and 200 μg of RNA were loading onto 10%-50% sucrose gradient (20 mM Tris-Cl pH 7.5, 5 mM MgCl₂, 150 mM NaCl, 1 mM DTT, 100 μg/mL CHX) and centrifuged in a TH-641 rotor (Thermo Scientific) at 34000 rpm for 2 h at 4 °C. Gradients were fractionated and collected using a BioLogic LP Chromatography System (BIO-RAD). Protein in fractions was methanol-precipitated and resuspended in 2×Laemmi buffer (BIO-RAD) with 2-mercaptoethanol (BIO-RAD) and denatured at 70 °C for 10 min. For in-gel fluorescence assay, 15 μL denatured samples were loaded to 4-15% Mini-PROTEAN TGX Precast Protein Gels, 12-well and run for 30 min with 200V. The gel was rinsed twice in deionized waster and imaged by iBright FL1500 Imaging System (Invitrogen).

### Confocal Microscopy of Ribo-Snap Labeled Cells

Cells were plated on poly-D-Lys coated coverslip 24 h before labeling. After ribo-snap labeling, cells were fixed with 4% formaldehyde (v/v) in DPBS for 15 min, followed by 3×wash with DPBS. Coverslips were mounted on slides with ProLong Gold Antifade Mountant with DAPI (Invitrogen) and imaged by ZEISS LSM 800.

### Quantitative Image Analysis

Images were taken with ×40 objective lens and were directedly processed in Fiji as follows: (1) subtract background, (2) gaussian blur, (3) split channels. For DAPI channel, the images were then processed: (1) threshold-auto, (2) fill holes, (3) erode, (4) watershed, (5) erode, (6) analyze particles, to generate region of interests (ROIs). The green channel images were then measured with ROIs for average intensity. The individual intensity data points were then normalized to the mean of control groups.

### Virus Production

293T cells were plated at a density of 1.0×10⁶ cells/well in a 6-well plate. Cells were grown for 20 h after plating. The medium was changed 1 h before transfection. Transfection was performed using Lipofectamine 3000. For one well in a 6-well plate, the transfection mixture contains 0.5 μg transfer vector (Brunello library or single LentiCRISPRv2-sgRNA plasmid), 0.3 μg pSPAX2 and 0.2 μg pMG2.G, with 2 μL P3000, 3 μL Lipo3000 and 250 μL Opti-MEM (Gibco 31985070). The mixture was incubated at room temperature for 15 min and was then added dropwise to the cells. Virus was harvested 48 h after transfection. The virus-containing medium was collected and centrifuged at 300×g for 3 min and the supernatant was then passed through 0.45 μM SFCA filter. The filtered virus-medium was then aliquoted and stored at −80 °C.

### Knock Out Single Gene by Using LentiCRISPRv2

Lentivirus was produced as mentioned in virus production. sgRNA sequences used in this research are listed in Table 2. The virus was transduced into HEK293T or HCT116 cells by mixing 10⁶ cells with 1 mL viral medium with 8 μg/mL polybrene in a total of 2 mL. The mix was plated onto 1 well in a 12 well plate. 24 h after transduction, cells were dislodged, counted and plated in complete medium with 2 μg/mL puromycin (HEK293T) or 1 μg/mL puromycin (HCT116) at 5.5×10⁵ cells/well in 6 well plate for a 6-day selection.

Pooled sgRNA Screen

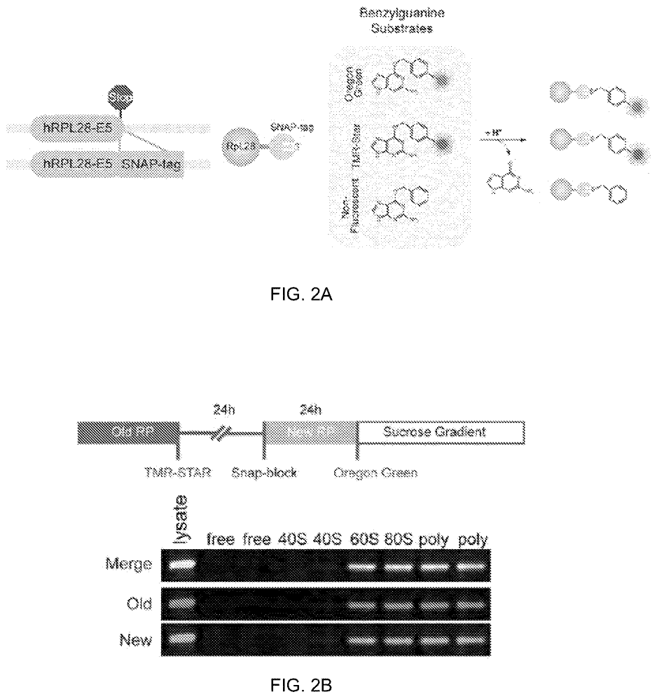

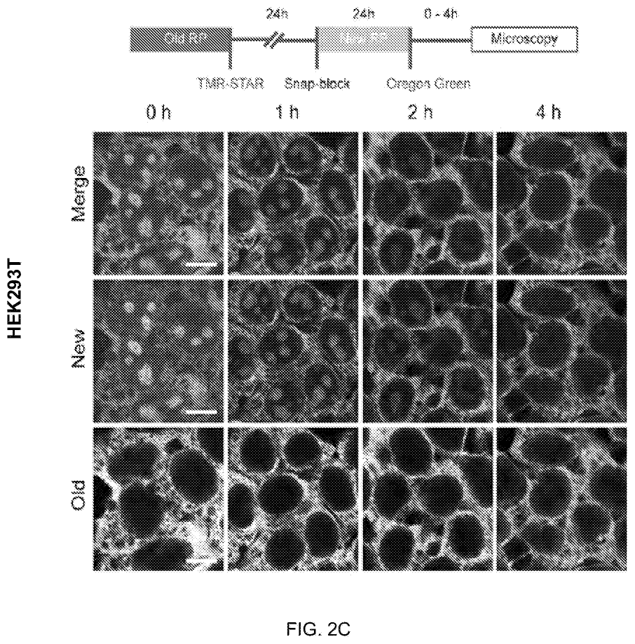

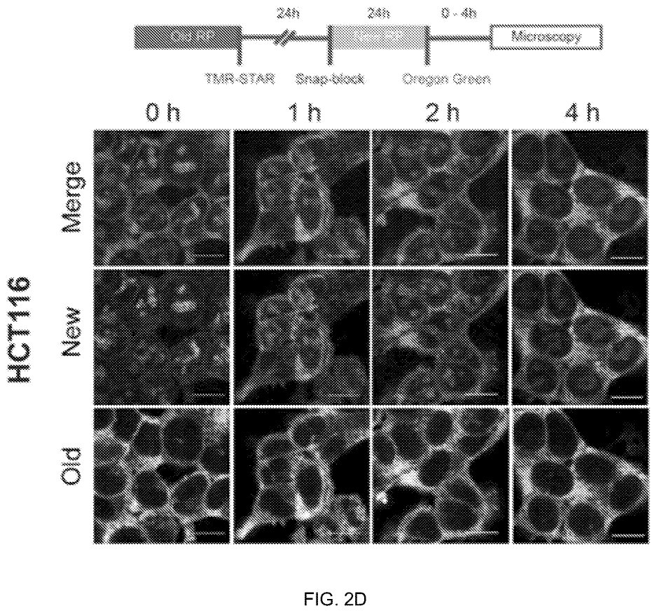

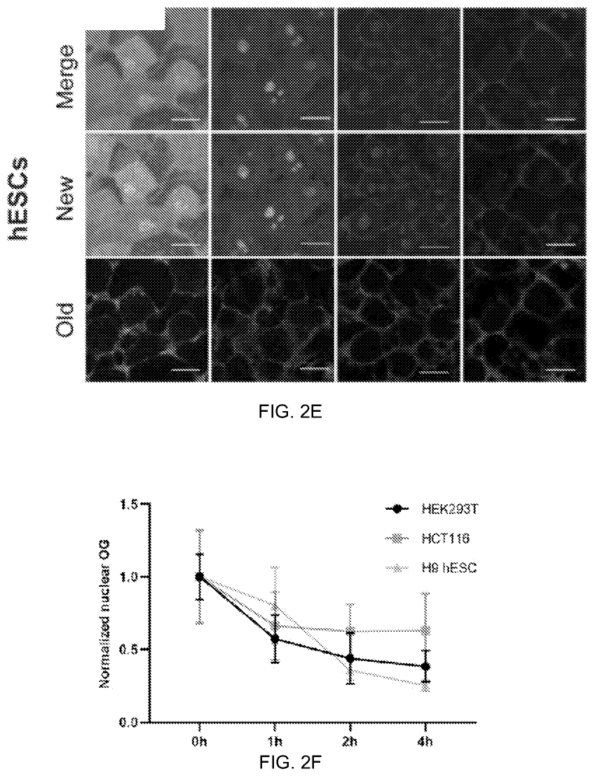

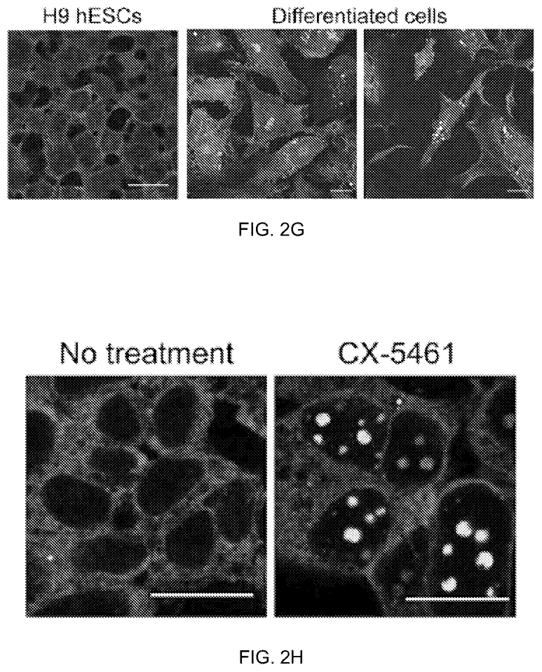

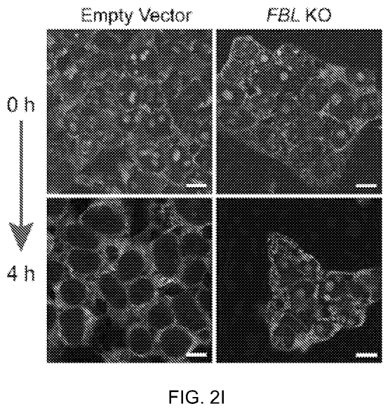

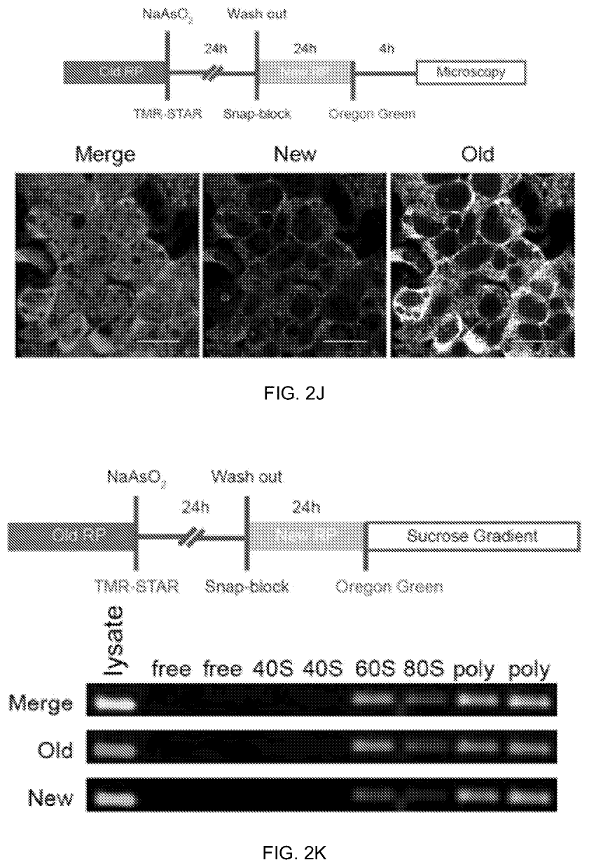

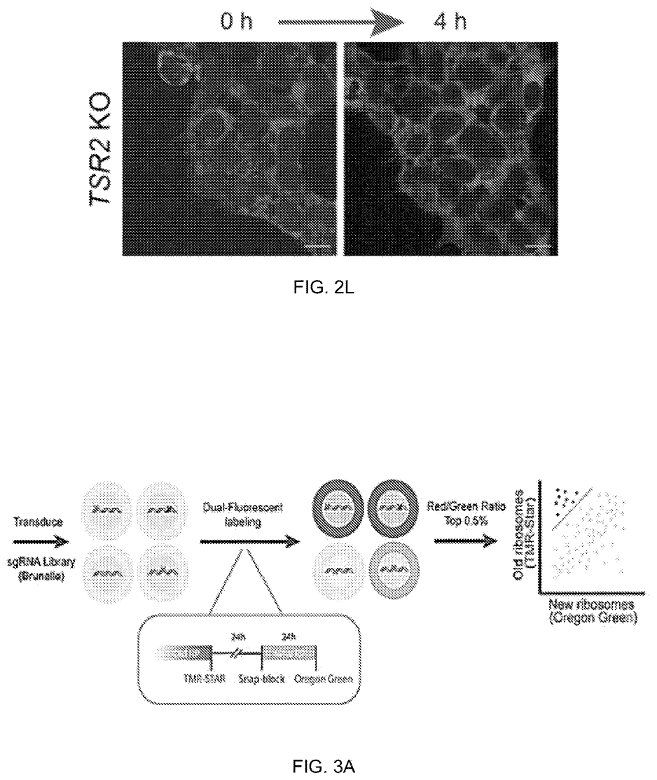

Lentivirus was produced as mentioned in virus production. The library was transduced by spinfection into 1.6×108 HEK293T.24 h after the end of spinfection, cells were dislodged, pooled and plated in complete medium with 2 μg/mL puromycin for a 7-day selection. On day 8, cells were dislodged, pooled, counted and then labeled sequentially by snap cell TMR-STAR (3 μM), snap cell Block (20 μM), snap cell Oregon Green (5 μM) as indicated in FIG. 2B. 4 h after snap cell Oregon Green labeling, cells were dislodged and resuspended in DPBS+2% FBS for sorting. Approximately 8×10⁷ cells were sorted at University of Texas Southwestern Moody Foundation Flow Cytometry Facility using a FACS Aria SORP (BD Biosciences). Cells with top 0.5% red/green ratio were collected. Approximately 3×10⁵ sorted cells and 4×10⁷ unsorted cells were pelleted and frozen at −80 °C until genomic DNA preparation. Sorted cells were lysed in STE buffer (10 mM Tris pH 8.0, 100 mM NaCl, 1 mM EDTA, 0.4% SDS, 0.2 mg/mL Proteinase K). 5 μL RNaseA (2 mg/mL) was added after Proteinase K reaction. Lysate were then separated by phenol-chloroform-isoamyl alcohol (PCI) and the aqueous layer was collected for ethanol precipitation. Precipitated DNA was resuspended in 30 μL H₂O. Unsorted cells were lysed in Tissue & Cell Lysis Solution (Lucigen) and the protein was removed by centrifuge after adding MPC Protein Precipitation Solution. The supernatant was collected, precipitated by isopropanol, and resuspended by 1.2 mL H₂O/3×10⁷ cells. Guides were amplified from genomic DNA by ExTaq following the protocol from previously published work (Sanson et al., 2018). PCR product was purified by AMPure XP. Purified libraries were sequenced by NextSeq® 500 (Illumina). NGS reads were analyzed by following MAGeCKFlute pipeline (Li et al., 2014; Wang et al., 2019) (See Supplemental Report).

### Flow Cytometry of Ribo-Snap Labeled Cells

Cells were grown in 24 well plate to maintain 20%-80% confluency during ribo-snap labeling. After labeling and chasing, cells were dislodged and resuspended in flow buffer (2% FBS in DPBS). The suspension passed through 70 micron cell strainer (FALCON). Cells were analyzed by flow cytometry using a LSRFortessa (BD Biosciences). Data were analyzed using Cytobank (Kotecha et al., 2010).

### AHA Incorporation and Click Chemistry for Protein Synthesis Assay

Cells were grown to ~80% confluency at the time of assay. Complete medium was replaced by AHA medium formulated with DMEM, high glucose, no glutamine, no methionine, no cystine (Gibco), 10% dialyzed FBS (Gibco), 1% GlutaMAX (Gibco), 1% Penicillin-Streptomycin (Gibco), 62.6 mg/L L-Cysteine (Sigma), 250 μM L-Azidohomoalanine (AHA, Click Chemistry Tools). For non-AHA control, the AHA was replaced by 30 mg/L L-Methionine (Sigma). For Anisomycin (ANS) treatment, AHA medium was further added with 10 μg/mL Anisomycin (Cell Signaling). After 3 h of incubation, cells were scraped in cold DPBS, washed twice with cold DPBS and lysed in RIPA buffer. Protein concentration of the lysate was quantified by BCA and input protein concentrations were normalized to 2.1 mg/ml for downstream click chemistry. 50 μL of the normalized cell lysate was used for click reaction with biotin-Alkyne (Click Chemistry Tools) by using Click-&-Go Click Chemistry Reaction Buffer Kit (Click Chemistry Tools). The reaction mixture was then methanol precipitated and was resuspended in 2×Laemmi buffer (BIO-RAD) with 2-mercaptoethanol (BIO-RAD) and denatured at 95 °C for 10 min. Final protein concentration is 1 mg/mL. The sample was then resolved by SDS-PAGE, and total protein signal was detected by One-Step Blue Protein Gel Stain (Biotium). The biotin signal was detected by HRP-Conjugated Streptavidin (Thermo Scientific).

### Crosslinking Immunoprecipitation

HEK293T cells were transfected with or without pCW57.1-C1ORF109-FLAG vector by using Lipo3000. Mock transfected and C1orf109-FLAG transfected cells were grown to 80% confluency in 100 mm dish. Cells were washed once in DPBS and were incubated with DPBS with 2 mM DSP (Thermo Scientific) for 30 min at room temperature. Crosslinking reaction was quenched by adding stop solution (1M Tris-Cl, pH 7.5) to a final concentration of 20 mM. Cells were lysed in FLAG-IP buffer (50 mM Tris-Cl pH 7.5, 150 mM NaCl, 1% Triton X-100, 1×complete Protease Inhibitor Cocktail). After lysis, lysate was centrifuged at 15000 g for 15 min at 4 °C to remove insoluble components. Protein concentration of the lysate was quantified by BCA and input protein concentrations were normalized to 0.5 mg/mL with FLAG-IP buffer. An aliquot from each sample was saved as input sample and ~500 μg total protein was incubated with 50 μL FLAG affinity gel (Sigma) for 2 h at room temperature. After incubation, samples were washed 2 times with 1×TBS, 2 times with FLAG-IP buffer. Samples were then eluted using FLAG-IP buffer with 500 μg/mL FLAG peptide (Sigma) at room temperature for 30 min. Samples were denatured with 4×Laemmi buffer (BIO-RAD) with 2-mercaptoethanol.

### Nuclear and Cytoplasmic Partitioning

Cells were grown to 80% confluency in 100 mm dish. Cells were washed twice in DPBS and were then lysed on ice by adding 600 μL/dish lysis buffer (20 mM Tris-Cl pH 7.5, 5 mM MgCl₂, 150 mM NaCl, 1×complete Protease Inhibitor Cocktail, 0.1% NP-40). Lysate was collected into 1.5 mL tube with 15×pipetting. A small aliquot of lysate was checked on microscope to ensure complete lysis of cells without disrupting nucleus. Lysate was then centrifuged at 4700 g for 30 s to pellet nuclei. The supernatant was transferred into a new tube and was the centrifuged at 21130 g at 4 °C for 10 min and the supernatant was collected as “cytoplasmic fraction”. The pelleted nuclei were washed once with DPBS and then lysed in 200 μL RIPA buffer with 3×sonication.

### Ribosome Pulldown

Cells were grown to 80% confluency in 100 mm dish. Cells were washed once in DPBS and were then lysed on ice by adding 500 μL/dish lysis buffer (20 mM Tris-Cl pH 7.5, 5 mM MgCl₂, 150 mM NaCl, 1 mM DTT, 1×complete Protease Inhibitor Cocktail, 0.1% NP-40). Lysate was collected into 1.5 mL tube and stay on ice for 15 min. Lysate was then centrifuged at 21130 g at 4 °C for 10 min to remove nuclei, mitochondria, and debris. RNA concentration of lysates was quantified by nanodrop and diluted to 150 ug/mL. 50 μl of lysate from each sample was saved as cytoplasmic ribosome input. 600 U RNaseT1 (Thermo Scientific) was added to 150 μg RNA and then mixed with 100 uL SNAP-Capture Magnetic Beads (NEB). SNAP-Capture Magnetic Beads was pre-washed by lysis buffer twice. The mixture was incubated at room temperature for 1 h and then 100 U SUPERaseln RNase Inhibitor (Invitrogen) was added to quench the reaction. The mixture was further incubated for 1 h at room temperature, for a total of 2 h incubation. After incubation, beads were washed 3 times with lysis buffer and then eluted with 50 μL 2×Laemmi buffer with 2-mercaptoethanol at 95 °C for 10 min.

### Northern Blot Analysis of Pre-rRNA

Total RNAs were purified with Trizol reagent and the RNA pellet was dissolved in RNase free water. RNA concentration was quantified by nanodrop and then diluted to 1600 ng/μL. 10 μg of RNA for each sample was used for pre-rRNA analysis. The northern blot was performed by using NorthernMax kit (Invitrogen). Probes that were used in this study are listed in Table 2.

### 5-EU Incorporation Assay for RNA Synthesis

Cells were plated on poly-D-Lys coated coverslip 24 h before labeling. On the time of the assay, cells were incubated with complete medium with 0.5 mM EU for 1 h. Cells were then washed twice with DPBS, fixed with 4% formaldehyde (v/v) in DPBS for 15 min and permeabilized with 0.5% Triton X-100 in DPBS for 15 min. After permeabilization, cells were washed once with 3% BSA in DPBS, and then incubated with click-reaction cocktail made with Click-&-Go Cell Reaction Buffer Kit and 2.5 UM AFDye 488 Azide (Click Chemistry Tools) for 30 min. After reaction, cells were washed once with 3% BSA in DPBS and once with DPBS. Coverslips were mounted on slides with ProLong Gold Antifade Mountant with DAPI and imaged by ZEISS LSM 800. Random regions were imaged, and the quantification is as described in “Quantitative image analysis”.

### Immunofluorescence

Cells were grown, fixed and washed the same as described in “Confocal microscopy of ribo-snap labeled cells”. After DPBS wash, cells were permeabilized with 0.1% Triton X100 in DPBS for 10 min then then incubated with blocking buffer (5% BSA, 0.1% Tween-20 in DPBS) for 60 min. Primary antibodies were diluted in antibody dilution buffer (1% BSA, 0.3% Triton X-100 in DPBS) and samples were incubated with antibodies at 4 °C overnight. After incubation, samples were washed 3 times with DPBS and incubated with secondary antibodies with antibody dilution buffer for 60 min at dark. Samples were then washed 3 times with DPBS and the coverslips were mounted on slides with ProLong Gold Antifade Mountant with DAPI.

### Transgenic Rescue

Cells were transduced by lentivirus and selected by puromycin as described in “Knock out single gene by using LentiCRISPRv2”. On day 4 of the selection, cells were transfected with rescue vectors by using Lipo3000. 24 h after transfection, cells were dislodged and plated in complete medium with puromycin for downstream assays.

### Phylogenetic Analysis

Homologs of CINP, C1ORF109, SPATA5 and SPATA5L were collected using BLAST and aligned using Muscle (Edgar, 2004). All positions with more than 25% gaps were eliminated after alignment. Evolutionary analyses were conducted in MEGA X. The evolutionary history was inferred by using the Maximum Likelihood method with the LG model. The tree with the highest log likelihood is shown, and branch confidence values (500 bootstrap replicates) are shown. Branch lengths are measured in the number of substitutions per site.

### Statistics

All statistical data were calculated using GraphPad Prism 8. Details on number of repeats (n) and the statistical test applied can be found in figure legends.

### Example 2: Heterochronic Labeling of Ribosomes in Different Cell Types

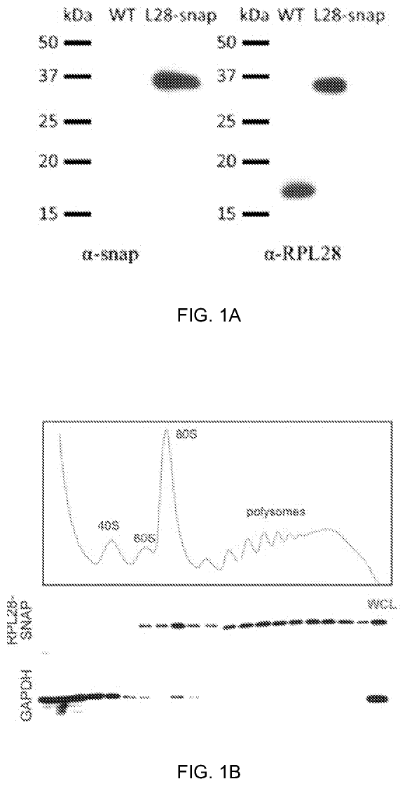

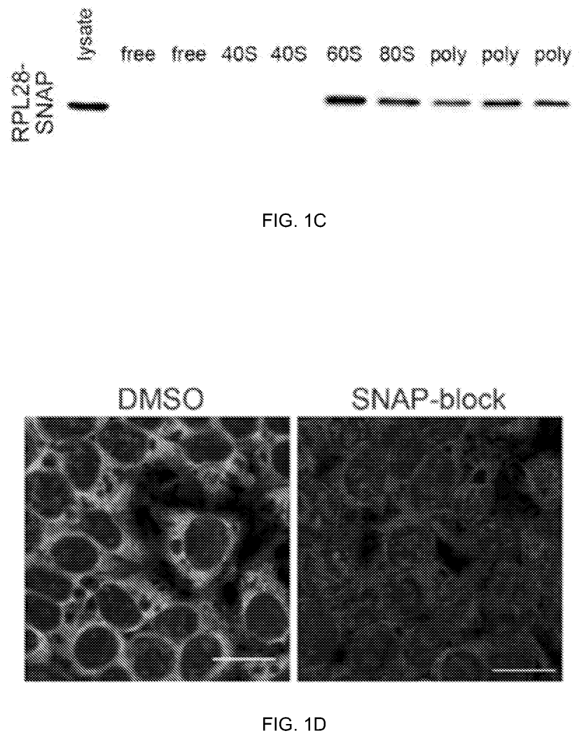

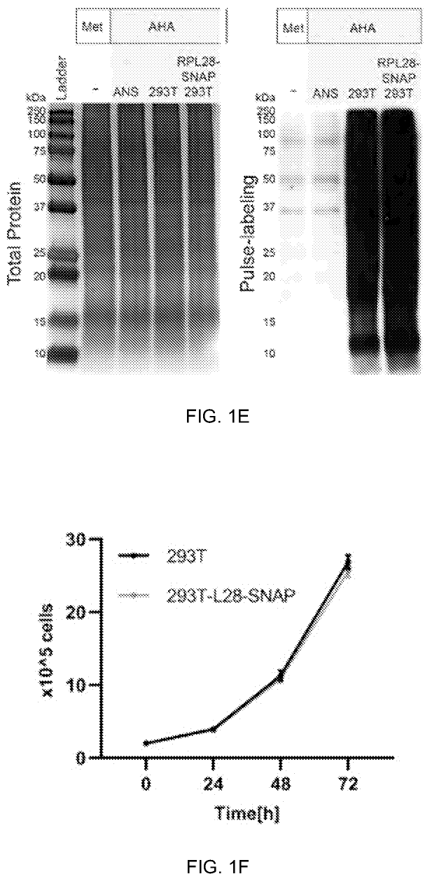

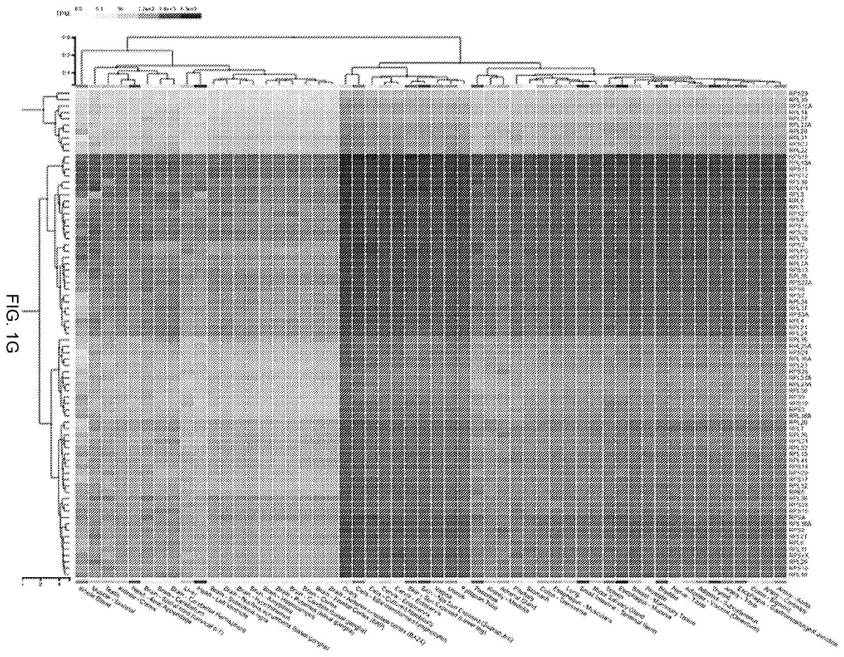

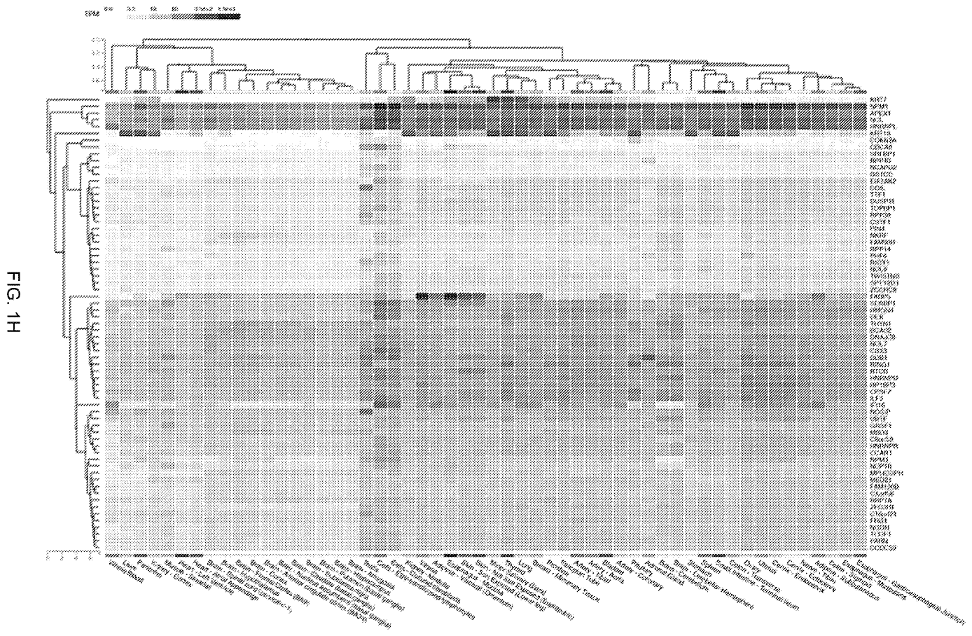

To characterize ribosome assembly dynamics under different conditions, SNAP-tag was incorporated into the endogenous Ribosomal Protein L28 (RPL28) gene in HEK293T, HCT116, and H9 human embryonic stem cells (hESCs) using CRISPR-based strategies (FIG. 1). The SNAP-tag is a self-labeling enzymatic tag, engineered from the human O-6-methylguanine-DNA methyltransferase that can form a covalent link with various fluorophores in living cells. Using sucrose gradient fractionation, it was found that SNAP-tagged RPL28 incorporated into the 60S subunit and readily contributed to monosomes and polysomes (FIGS. 1A-1C). Moreover, proliferation rates and global translation after incorporation of the tag match those of parental controls (FIGS. 1D-1F).

Three different SNAP-tag substrates were sequentially applied (TMR-star (red fluorescent), non-fluorescent, and Oregon green (green fluorescent)) to separately label pre-existing and newly produced ribosomes (FIGS. 2A-2C). The SNAP-tag performs a suicide reaction and cannot react with other substrates after its first labeling, which ensures that red and green labeling mark chronologically distinct ribosome populations. Generally, a strategy was used whereby red-labeled ribosomes were at least 48 hours old and green-labeled ribosomes were 0-24 hours old. Assaying fluorescently labeled old and new ribosomes using sucrose gradient fractionation confirmed the rapid incorporation of newly labeled RPL28 into functional 60S subunits, 80S monosomes, and polysomes under normal conditions (FIG. 2B). Moreover, the relative levels of old and new ribosomes appeared similar across the 80S and polysome fractions. These results suggest ribosomes that are at least 48 hours old are functionally equivalent to newly synthesized ribosomes.

As expected, red-labeled old RPL28 resided in the cytoplasm while green-labeled new RPL28 was observed in nucleoli immediately after the labeling procedure. Subsequent chase periods showed that the vast majority of nucleolar RPL28 was rapidly exported out into the cytoplasm within 1 hour of labeling (FIGS. 2C-2E). These levels continued to drop over the subsequent 3 hours. The rate at which newly labeled RPL28 trafficked into and out of the nucleolus differed between HEK293T, HCT116, and hESCs (FIGS. 2C-2F). Moreover, pulse-labeling revealed different levels of newly labeled RPL28 in the nucleoli of hESCs and their differentiated progeny (FIG. 2C-2E and FIG. 2G). Levels of old ribosomes also differed between these cells, likely reflecting differences in cell division rates or ribosome turnover. RNA sequence data from the genotype-tissue expression (GTEx) database shows that expression levels of different ribosome biogenesis factors vary greatly between different tissues, especially when compared to translation factor and ribosomal protein mRNAs (FIG. 1G). These results suggest that ribosome assembly rates are regulated at multiple levels to control the total number of functional ribosomes within a cell.

### Example 3: Heterochronic Labeling Reveals Perturbations in Ribosome Assembly and Function

How perturbations in ribosome production and activity influenced the behavior of old and newly labeled RPL28 was tested next. Ribosome biogenesis depends on ongoing rRNA transcription. Treating cells with the Pol I and TOP2B inhibitor CX-5461 resulted in retention of newly labeled RPL28 within nucleoli (FIG. 2H), further suggesting that trafficking of RPL28 from the nucleolus to the cytoplasm depends on ribosome assembly. Treatment of cells with sodium arsenite (NaAsO₂) results in stress granule assembly and a rapid loss of protein synthesis. To see if NaAsO₂ exposure alters 60S subunit use, preexisting ribosomes were labeled with TMR-star and these cells were exposed to NaAsO₂ for 24 hours (FIG. 2J and FIG. 2K). The cells were then washed, and ribosomes formed after the treatment were labeled with Oregon green. While NaAsO₂ exposed ribosomes were found in polysome fractions, they also accumulated in the 60S fractions and were reduced in the 80S fraction. By contrast, ribosomes assembled after NaAsO₂ treatment were largely incorporated into polysomes. These results suggest that newly made 60S subunits rapidly substitute for their damaged counterparts to support the ongoing demands for translation elongation competent ribosomes.

Next, the dynamic behavior of new and old ribosomes in different mutant backgrounds was examined. Disruption of ribosome assembly factors or rRNA processing machinery would be predicted to slow down or halt specific aspects of ribosome biogenesis, potentially leading to the retention of new RPL28 within nucleoli. To test this, two ribosome biogenesis factors were targeted: Fibrillarin (FBL) and TSR2 Ribosome Maturation Factor (TSR2). FBL is an essential rRNA 2′-O-methyltransferase that acts at an early step in ribosome biogenesis, whereas TSR2 promotes the release of the small subunit protein RPS26 from Importin upon entry into the nucleus and participates in 20S pre-rRNA processing. TSR2 has been linked with different ribosomopathies, including Diamond-Blackfan Anemia. CRISPR-mediated mutation of FBL resulted in prolonged retention of RPL28 within nucleoli, even after a 4-hour chase period, reflecting defects in ribosome assembly and subsequent defects in pre-60S export out of the nucleus (FIG. 2B and FIG. 2I). By contrast, knockout of TSR2 resulted in a reduction of new RPL28 levels, as reflected by the loss of Oregon green signal, but did not cause retention of nucleolar RPL28 (FIG. 2L). Thus, as expected, loss of TSR2 does not appear to interfere with ongoing assembly and trafficking of pre-60S subunits. Of note, FBL or TSR2 mutant cells retain higher levels of mature ribosomes in the absence of new ribosome production.

### Example 4: CRISPR Screen Reveals Regulators of Ribosome Biogenesis

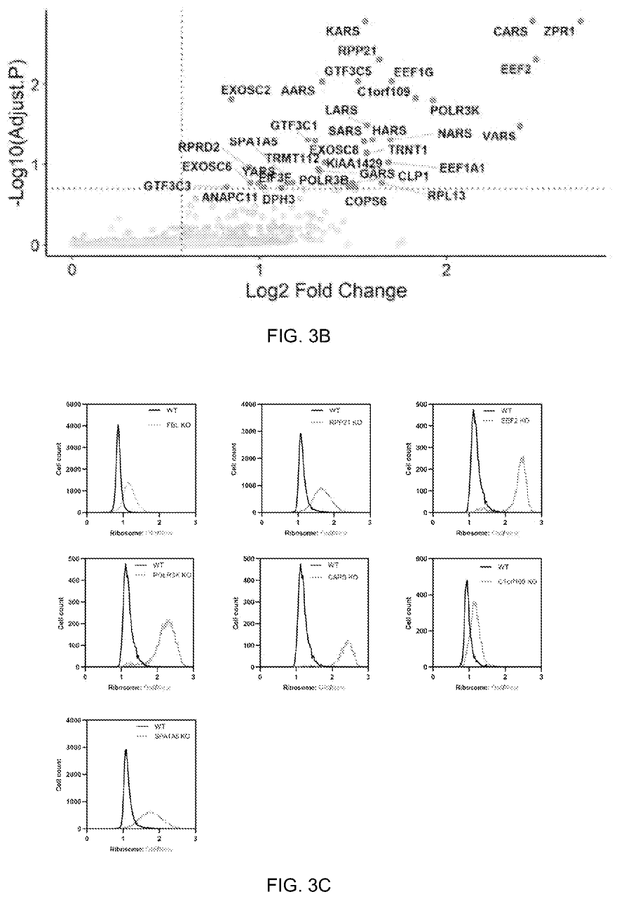

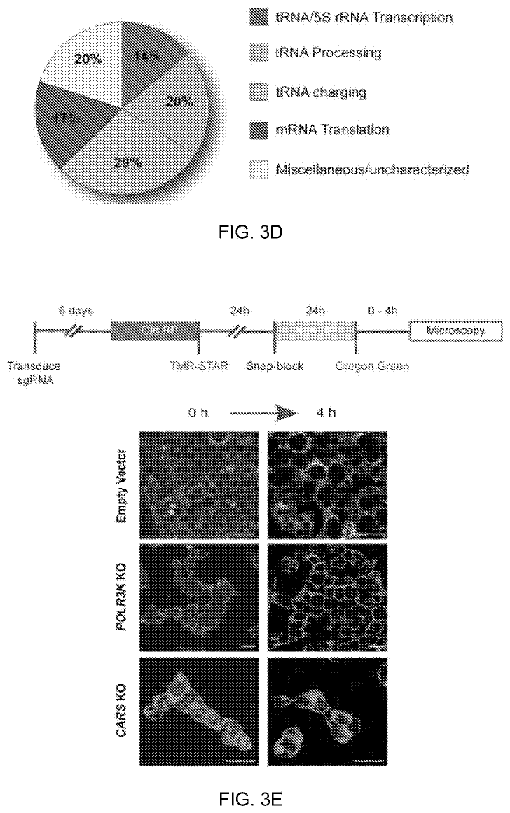

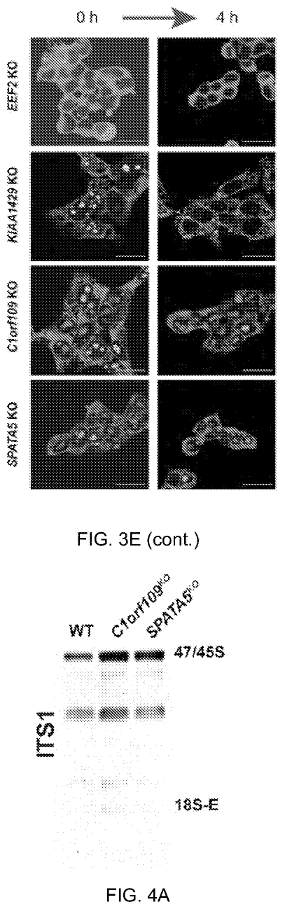

A loss-of-function CRISPR-based screen was conducted to gain insights into the regulatory landscape that controls ribosome levels in rapidly dividing cells, using the SNAP-tagged RPL28 HEK293T cell line (FIG. 3A). Through the course of these studies, it was found that knockout of core ribosome assembly factors resulted in cell lethality 4-6 days after transduction of sgRNA expression. To identify new genes that control ribosome biogenesis levels, the selection was limited to 8-days following delivery of the CRISPR library. Using the Human CRISPR Knockout Pooled Library (Brunello), RPL28-SNAP cells were transduced and selected for expression of sgRNAs. Starting on day 6 after transduction, old (>=48 h) and new (<=24 hrs) ribosomes were pulse-labeled with red and green fluorophores, respectively. Cells within the top 0.5% of the “brighter red and dimmer green” population were sorted. The experiments were specifically focused on hits that exhibited less green fluorescence and more red fluorescence, reasoning that this shift in old and new ribosomes would reveal new factors that regulate ribosome biogenesis. Next generation sequencing (NGS) was used to verify high overall representation of the entire library in the initial population of infected cells. sgRNAs present within the sorted cell populations were identified using NGS and this data was analyzed by MAGeCK. This analysis revealed 35 high-confidence hits (FIG. 3B-3D), including multiple aminoacyl-tRNA synthetases (CARS, KARS, AARS, LARS, VARS, NARS, HARS, and SARS), tRNA/5S rRNA transcription and processing factors (GTF3C5, GTF3C1, POLR3K, and RPP21), multiple components of the RNA exosome (EXOSC2 and EXOSC8), and genes involved in translation elongation (eEF1A, eEF1G and eEF2). Cell imaging and flow cytometry confirmed that loss of representative genes results in both loss of new ribosome labeling and a retention of old ribosomes, relative to control cells (FIGS. 3B-FIG. 3D).

### Example 5: C1Orf109 and SPATA5 Control a Late Step in Ribosome Maturation

During screen validation efforts, fluorescence microscopy revealed that newly labeled RPL28 persisted in the nucleoli of C1orf109 and SPATA5 mutant cells for over 4 hours (FIG. 3E). C1ORF109 has been linked with cell proliferation, but little is known about its molecular function. SPATA5 belongs to the AAA ATPase family of proteins and is distantly related to the yeast protein Drg1 (also known as AFG2). AAA ATPases play important roles in the assembly of ribosomes by using ATP hydrolysis to drive changes in protein-protein interactions and protein conformation. For example, during ribosome assembly in yeast, RLP24 serves as a placeholder within the pre-60S ribosomal subunit. Once the pre-60S subunit has been exported to the cytoplasm, Drg1 helps to remove RLP24, allowing for recruitment of RPL24 to the pre-60S subunit. Drg1 performs this function as a single protein. A recent study characterized the cryo-EM structure of Drg1 and suggested potential homology between this protein and SPATA5.

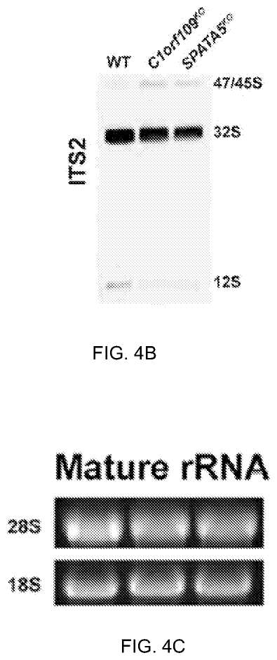

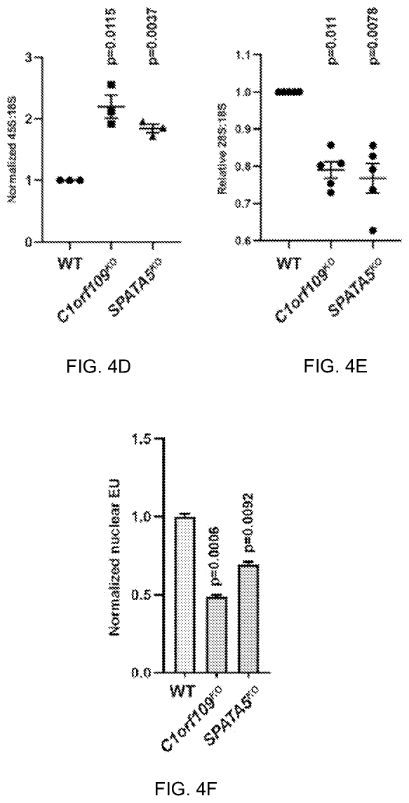

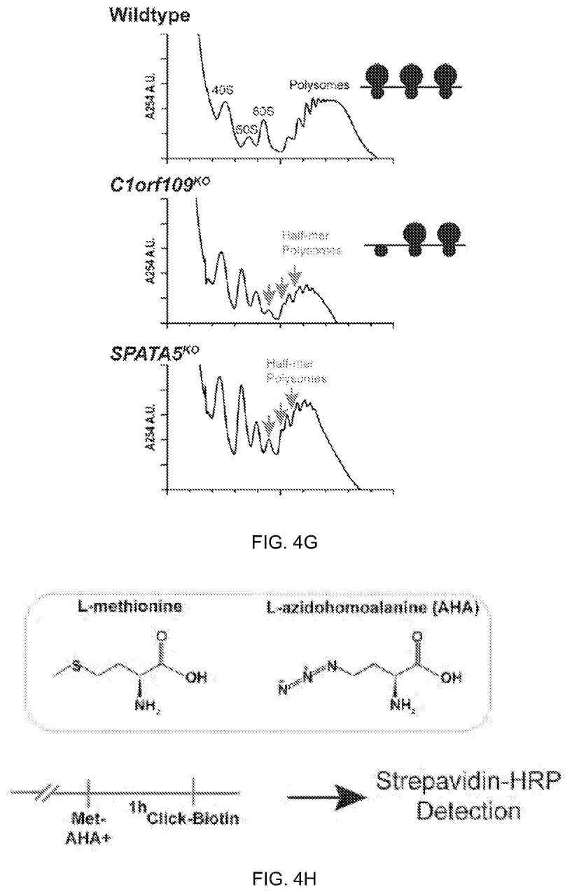

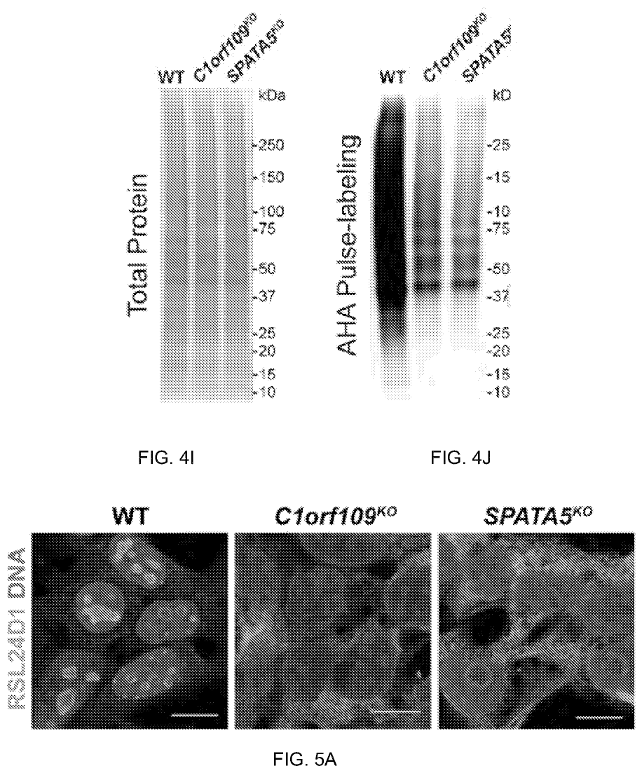

Given their shared RPL28 nucleolar retention phenotypes, it was reasoned that C1ORF109 and SPATA5 may work together during ribosome biogenesis. To investigate the function of these genes further, rRNA processing and transcription in C1orf109 and SPATA5 mutant cells was assayed. Northern blot analysis of total RNA using a combination of ITS1 and ITS2 probes (SEQ ID NOS: 22 and 23) that distinguish different intermediate processed forms of rRNA were used (FIGS. 4A-4C). These experiments revealed that loss of C1orf109 and SPATA5 result in an increase of unprocessed 47S/45S pre-rRNA and a reduction of 12S pre-rRNA. A decrease in the ratio of mature 28S to 18S was also observed (FIGS. 4A, 4D and 4E), which suggests a potential defect in 60S maturation. Subsequent 5-Ethynyl Uridine (EU) pulse-labeling experiments indicated that disruption of C1orf109 and SPATA5 both resulted in a reduction of labeled nascent nucleolar/nuclear RNA (FIGS. 4D and 4E), indicating that the increased levels of 47S/45S rRNA in C1orf109 and SPATA5 mutant cells observed by northern blot reflect a processing defect and not increased rRNA transcription.

To further assess the impact of C1orf109 and SPATA5 loss on ribosome biogenesis and function, polysome profiles from wild-type and mutant cells were examined. These experiments revealed that C1orf109 or SPATA5 mutant cells exhibit an imbalance between the 40S and the 60S subunits and formation of half-mers, which represent polysomes with stalled 48S initiation complexes (FIG. 4F). Half-mers are commonly observed in mutants that exhibit reduced levels of functional 60S subunits. The appearance of half-mers in C1orf109 and SPATA5 mutant extracts suggest that these two proteins promote pre-60S maturation.

Since both reduction of ribosome maturation and formation of half-mers could be the indicators of impaired mRNA translation, a series of pulse labeling experiments using azidohomoalanine (AHA) to test if loss of C1orf109 or SPATA5 leads to impaired global protein synthesis were performed. Activation of AHA by tRNAMet-synthetase allows azides to become incorporated into proteins, which can then be labeled through click chemistry (FIG. 4G). Pulse-labeling control and mutant samples revealed that loss of C1orf109 and SPATA5 resulted in lower levels of translation, as expected for proteins needed for ribosome biogenesis (FIGS. 4H-4J).

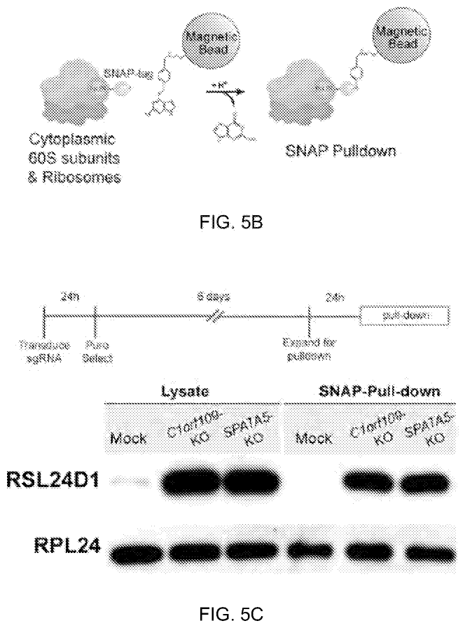

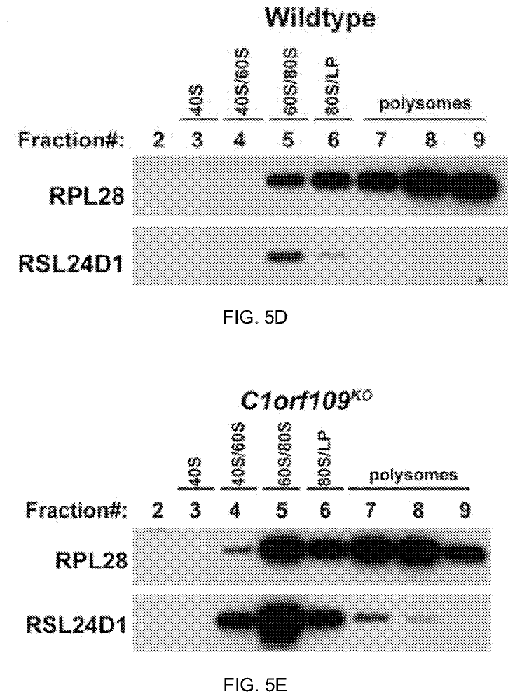

Given the distant sequence homology between Drg1 and SPATA5, whether loss of either C1orf109 or SPATA5 results in retention of RSL24D1 (human ortholog of RLP24) on pre-60S subunits was tested. Strikingly, the sub-cellular localization of RSL24D1 shifted from the nucleolus to the cytoplasm in C1orf109 and SPATA5 mutant HEK293T cells (FIG. 5A), suggesting that the recycling of RSL24D1 is disrupted in the absence of these two factors. Next, whether cytoplasmic RSL24D1 continued to associate with pre-60S ribosomal subunits was examined. A SNAP pulldown was employed, whereby cytoplasmic lysates from control and mutant cells expressing SNAP-tagged RPL28 with benzylguanine beads were incubated (FIG. 5B). Consistent with the immunofluorescent analysis, the levels of RSL24D1 in cytoplasmic lysates increased in C1orf109 and SPATA5 mutant cells (FIG. 5C). Moreover, high levels of RSL24D1 precipitated with SNAP-tagged ribosomes, indicating that this protein remains inappropriately associated with cytoplasmic pre-60S subunits in the absence of C1orf109 and SPATA5 (FIG. 5C).

To further characterize the behavior of RSL24D1 and its association with the pre-60S subunit, sucrose gradient fractionation in wildtype and mutant cells was performed. In wildtype cells, RSL24D1 fractioned with pre-60S subunits and was not observed to fractionate with monosomes or polysomes (FIG. 5D). By contrast, RSL24D1 exhibited a dramatic increase in the fractions occupied by 60S subunits, monosomes and polysomes in SPATA5 and C1orf109 mutant cells (FIGS. 5E and 5F). However, RSL24D1 was absent from the heaviest polysome fractions in mutant cells, indicating that RSL24D1 containing pre-60S subunits likely do not participate in productive translation.

### Example 6: Disease-Linked Allelic Variants of SPATA5 Compromise Ongoing Ribosome Biogenesis

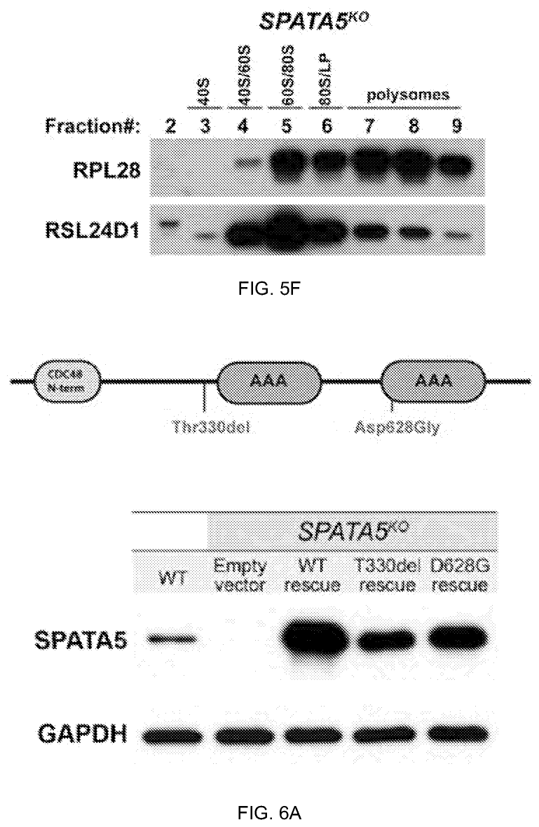

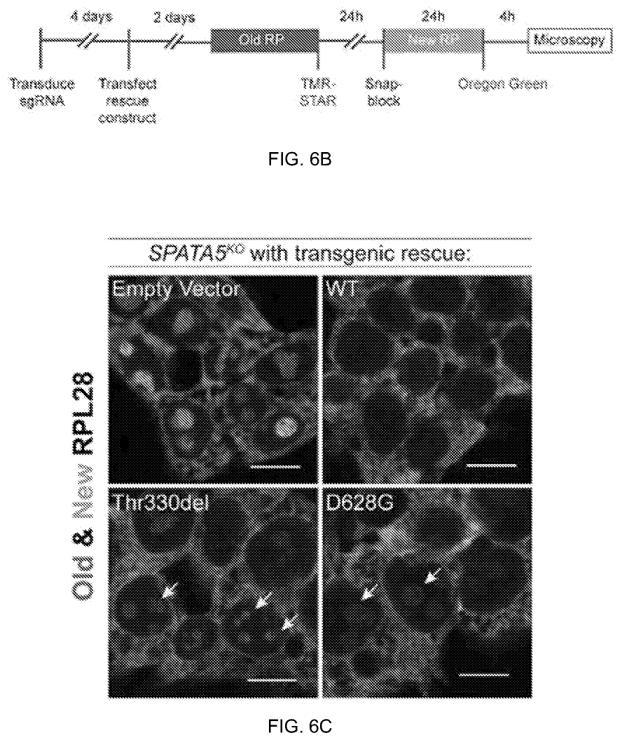

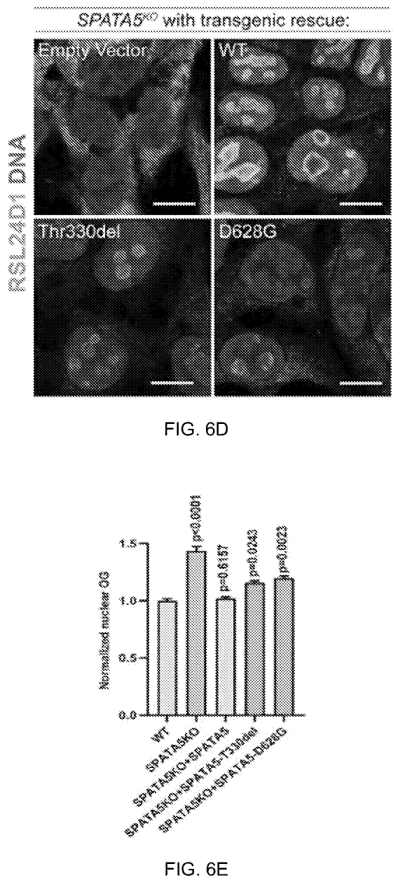

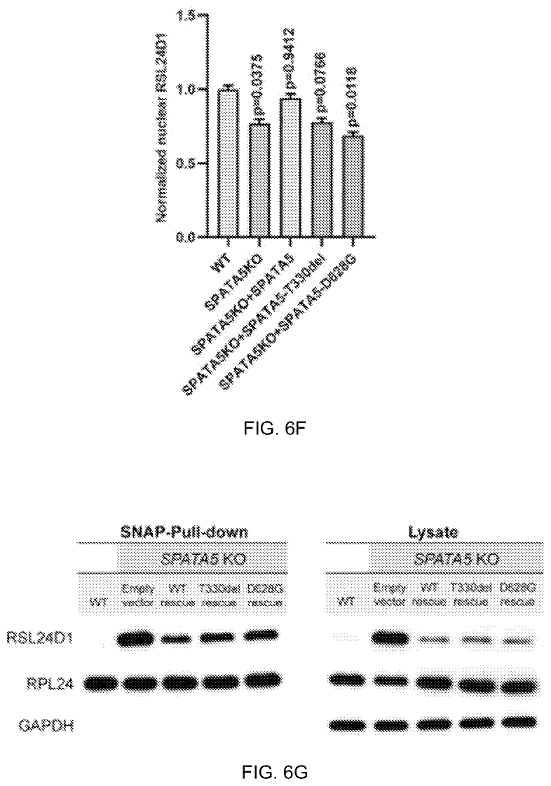

Allelic variants of SPATA5 in humans have been linked with several neurodevelopmental disorders including microcephaly, hearing loss, epilepsy, and intellectual disability. A previous study suggested SPATA5 phenotypes may be caused by changes in mitochondrial morphology and activity. However, data presented here indicates that SPATA5 plays an essential role in ribosome assembly, suggesting that pathogenic mutations may compromise ribosome biogenesis and subsequent protein synthesis. To test this hypothesis, the ability of wildtype and two variant transgenes (FIG. 6A) to rescue the SPATA5 knockout phenotypes were compared. RPL28 levels and localization was assayed, as well as the subcellular distribution of RSL24D1 (FIGS. 6B-6F). The expression of all three transgenes was higher than physiological SPATA5 levels (FIG. 6A bottom panel), but the two pathogenic variants were expressed at slightly lower levels, suggesting that these proteins may be less stable or subject to quality control. Labeling old and new ribosomes showed that while a wildtype SPATA5 transgene largely rescued the retention of newly labeled RPL28 within nucleoli of SPATA5 knockout cells, cells expressing the Thr330del and D628G human variants displayed low levels of nucleolar RPL28 even after a 4-hour chase period (FIGS. 6C and 6E). Moreover, a modest but significant increase of cytoplasmic and a reduction in nucleolar RSL24D1 upon rescue with the allelic variant transgenes compared to the wildtype control was observed (FIGS. 6D and 6F). Expression of both allelic variants also resulted in a modest increase in the amount of RSL24D1 that remained associated with cytoplasmic pre-60S subunits relative to the wildtype transgene (FIG. 6G). These data suggest that the Thr330del and D628G variants represent weak loss of function alleles of SPATA5 in the context of pre-60S subunit maturation.

### Example 7: C1Orf109 and SPATA5 Work Together with SPATA5L1 and CINP

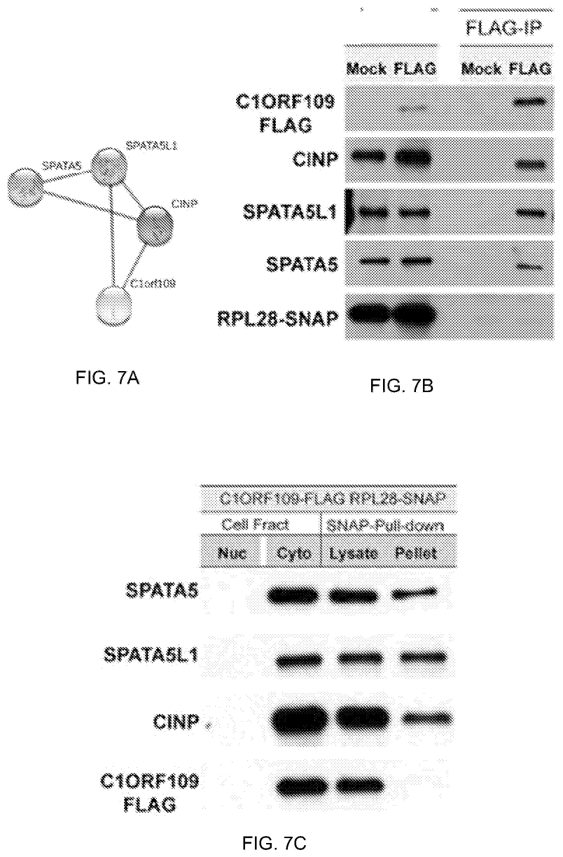

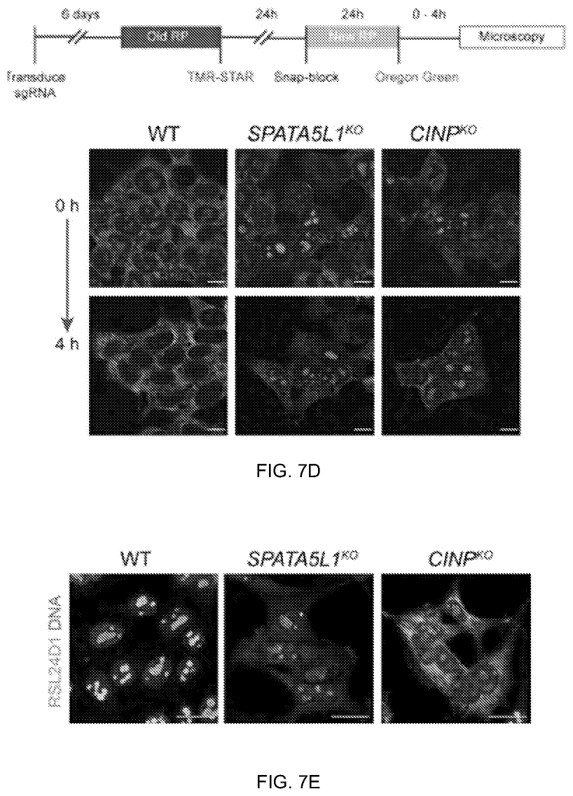

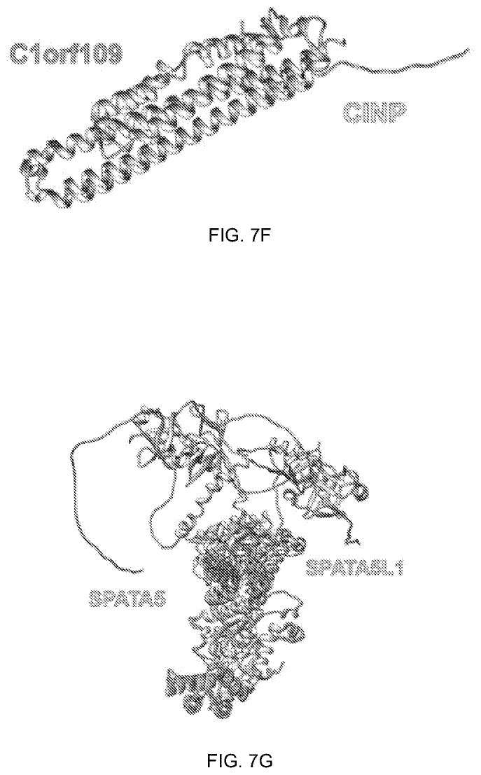

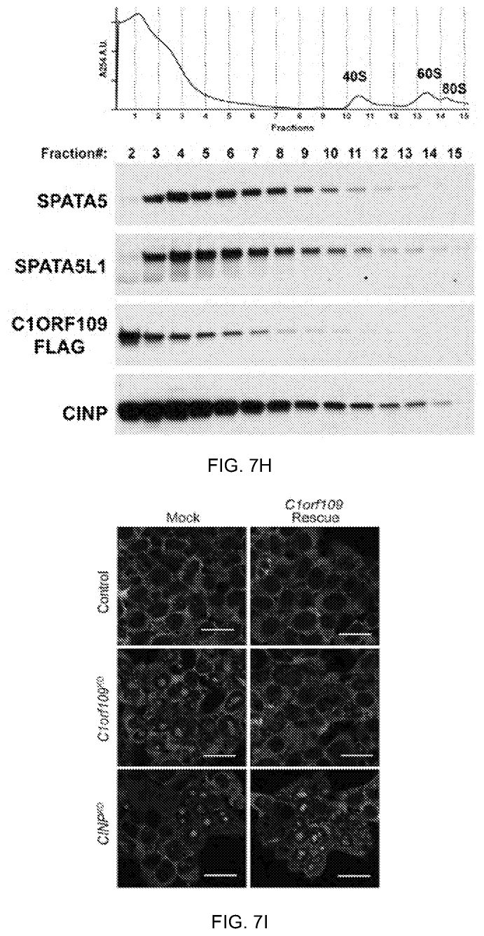

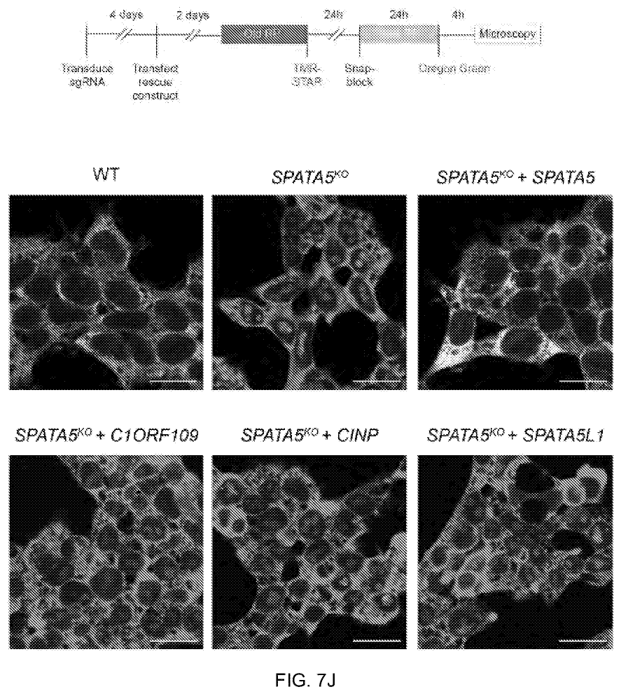

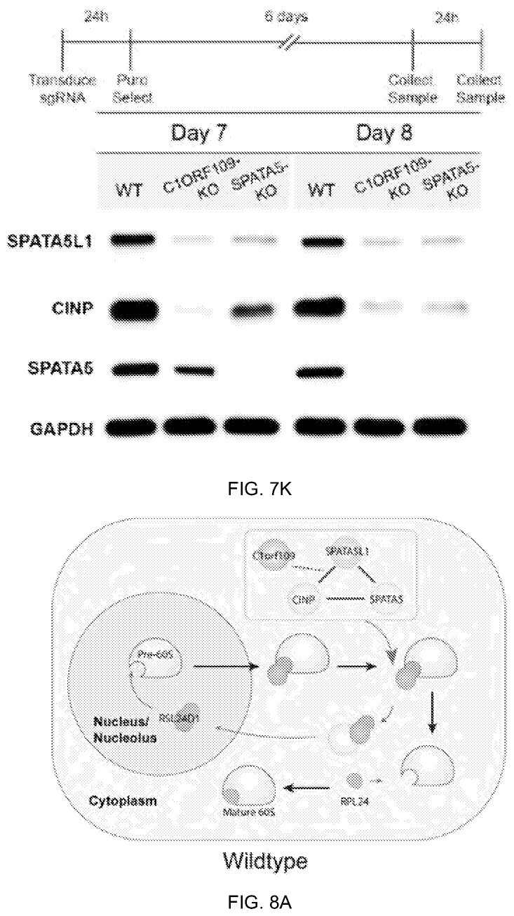

C1ORF109 and SPATA5 have been linked with two other proteins: SPATA5-like 1 (SPATA5L1) and Cyclin Dependent Kinase 2 Interacting Protein (CINP) (FIG. 7A left panel). SPATA5L1 is a second AAA ATPase of unknown function. CINP is a component of the DNA replication complex and functions to regulate checkpoint signaling in response to DNA damage. Immunoprecipitation experiments in the presence of DSP crosslinking was done and it found that C1ORF109 interacts with all three other proteins (FIG. 7B). To determine whether these four proteins interact with ribosomes, the SNAP-tagged based pull-down strategy using benzylguanine beads was performed. Ribosomes were precipitated from cells carrying SNAP-tagged RPL28 and FLAG-tagged C1ORF109. While SPATA5, SPATA5L1 and CINP physically interacted with ribosomes (FIG. 7C), C1ORF109 did not under the conditions tested, consistent with results from the C1ORF109 IP. However, loss of C1orf109 resulted in a substantial decrease in SPATA5, SPATA5L1, and CINP levels (FIG. 7K). Together, these data suggest that physical interactions between all four proteins help to stabilize them within cells. Similar to loss of C1orf109 and SPATA5, knockout of both SPATA5L1 and CINP resulted in retention of nucleolar RPL28 after 4 hours of labeling and a redistribution of RSL24D1 (FIGS. 7D and 7E), indicative of a ribosome biogenesis defect. However, this retention was much more sporadic across the population of cells, likely reflecting inefficiency of the specific guide RNAs used for Cas9 targeting and providing a reasonable explanation for why these genes were not identified in our initial screen.

To gain further insights into how SPATA5, SPATA5L1, CINP, and C1ORF109 cooperate to promote pre-60S maturation, their evolutionary history and predicted structures were compared. As mentioned above, SPATA5 and SPATA5L1 both belong to the AAA ATPase family of enzymes and share a common domain structure, including a CDC48 N-terminal domain followed by two AAA domains (FIG. 7F). Although related to one another, Alphafold predictions (Senior et al., 2020) highlight differences between the N termini of SPATA5 and SPATA5L1 (FIG. 7F), further suggesting these proteins carry out non-overlapping functions in removing RSL24D1 from pre-60S subunits. Phylogenetic analysis shows that CINP and C1ORF109 are likely related to one another. Orthologs of these proteins are not present in yeast, but CINP-like proteins are found in Drosophila and C. elegans, among other species. Matchmaker alignments of AlphaFold predictions indicate that CINP and C1ORF109 share remarkably similar structures (FIG. 7F), with the one notable difference being that CINP has a 14 AA N-terminal extension not found in C1ORF109. Given the similarities in their predicted structures, whether C1ORF109 and CINP could compensate for one another was tested. While C1ORF109 over-expression rescued the ribosome biogenesis defects in C1orf109 knockout cells, it did not rescue these same defects in CINP mutant cells (FIG. 7G). Similarly, over-expression of SPATA5L1, CINP or C1orf109 did not rescue phenotypes caused by loss of the SPATA5 suggesting these proteins carry out discrete functions during pre-60S maturation.

Next, sucrose gradient fractionation over a much steeper gradient followed by western blot analysis to further characterize potential higher order interactions between SPATA5, SPATA5L1, C1ORF109, and CINP was performed (FIG. 7H). SPATA5 and SPATA5L1 displayed a remarkably similar distribution across the gradient leading up to heavier fractions that contain 40S subunits, 60S subunits, and 80S monosomes. Both appeared largely absent from the first and lightest fractions. By contrast, C1ORF109 and CINP were both present in the lightest fractions. Like SPATA5 and SPATA5L1, CINP was distributed over a broad range of fractions, consistent with the observation that CINP physically associates with pre-60S subunits, while C1ORF109 was not. While these data suggest SPATA5, SPATA5L1, CINP, and C1ORF109 interactions may be varied and potentially dynamic, further biochemical and structural studies will be needed to more fully understand complex assembly and the specific roles that each protein plays in promoting the dissociation of RSL24D1 from pre-60S subunits.

### Example 8: Discussion

Ribosome biogenesis is generally considered a constitutive process, and current models have most regulation of ribosome production occurring at the level of rRNA transcription or the expression of ribosomal protein genes. Evidence for the dynamic regulation of multiple steps of ribosome assembly in a cell-specific manner is provided using the methods described herein. For example, the rates of both new ribosomal protein import into the nucleolus and pre-60S subunit export out of the nucleus differs between HEK293T cells, HCT116 cells, hESCs, and their differentiating progeny. Multicellular organisms have evolved additional ribosome biogenesis factors not present in yeast and other unicellular organisms. In addition, the expression levels of human ribosome assembly factors vary broadly across different tissues. These observations, combined with other results from this study, suggest that higher eukaryotes may have evolved many ways to fine tune particular steps of ribosome biogenesis to fit specific cellular requirements during development and stress response. The enhanced complexity of the ribosome assembly process potentially endows cells from multicellular organisms with the ability to modulate their protein synthesis capacity in ways distinct from their unicellular ancestors.

The catalog of factors that regulate and participate in ribosome biogenesis in humans and other multicellular organisms likely remains incomplete. Previous screening efforts have targeted specific genes based on biochemical identification of nucleolar proteins or visual criteria. These efforts revealed hundreds of human ribosome biogenesis factors. The list of high significance hits from the screens described herein shows little overlap with those identified previously, although these studies identified genes involved in common processes such as translation initiation. Novel genes were identified with no known link to ribosome biogenesis, including C1ORF109 using this method. Varying the timing of the pulse labeling assay after sgRNA transduction and screening additional cell types may lead to the discovery of additional ribosome biogenesis factors, particularly those that do not display enriched localization to nucleoli.

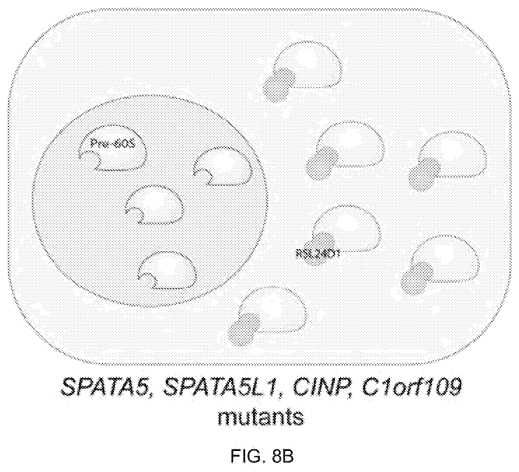

Work presented here provides insights into the cytoplasmic maturation of pre-60S subunits in human cells. More specifically, SPATA5, C1ORF109, SPATA5L1, and CINP were found to function together to promote the recycling of RSL24D1 from cytoplasmic pre-60S ribosomal subunits back to the nucleolus (FIGS. 8A and 8B). In yeast, this step is carried out by the single protein Drg1. While this data indicates that SPATA5 and SPATA5L1 physically interact with ribosomes and are both needed for the recycling of RSL24D1, a detailed understanding of how these two AAA ATPases work together, whether they form hexameric rings similar to yeast Drg1, whether such rings are homomeric, heteromeric or double ring structures, and how C1ORF109 and CINP foster the formation and activity of these potential complexes will require additional study.

This data suggests that defects in ribosome biogenesis and protein synthesis may underlie the phenotypes associated with SPATA5 variants in human patients. Rescue experiments provided herein show that two allelic variants of SPATA5 associated with disease in humans display minor defects in ribosome biogenesis. SPATA5, SPATA5L1, CINP, and C1orf109 mRNA expression levels appear low in many regions of the brain based on RNA sequence data from genotype-tissue expression (GTEx) databases, perhaps making these cells acutely sensitive to minor perturbations in late steps in cytoplasmic ribosome maturation. Allelic variants of human CINP and SPATA5L1 have also been linked with microcephalic and neurodevelopmental phenotypes, strikingly similar to those associated with SPATA5. SPATA5L1 variants have also been linked with autism. Mutations in ribosomal proteins and specific ribosome assembly factors result in a group of diseases, collectively known as ribosomopathies. While these disorders result in a range of phenotypes, they are most often associated with anemia and craniofacial defects. Work in several model systems suggests that these defects arise, at least in part, because of a nucleolar stress response and p53-induced cell death.

Pulse labeling ribosomal proteins allows one to trace the half-lives of ribosomes. Ribosome turnover varies between different cell types. In rapidly dividing cells, such as those examined in this study, old ribosomes quickly become diluted through cell division. The differentiated progeny of hESCs exhibit some differences in the levels of old ribosomes (FIGS. 2A-2L) and it will be of interest to examine ribosome half-lives more systematically in different post-mitotic cells. Initial analysis presented here suggests that ribosomes at least 48 hours old show the same ability to enter polysomes as newly generated ribosomes. Pulse-labeling strategies can be used to look for potential differences between heterochronic ribosomes over longer time scales and at higher resolution. Furthermore, though these examples focused on the biology of the pre-60S and mature 60S subunits, similar strategies can be used to study 40S subunit assembly and function. The further functional characterization of ribosome assembly dynamics and ribosome turnover promises to provide new insights into how translation machinery is regulated by diverse developmental processes across species and how perturbations in ribosome homeostasis contribute to a broad range of human diseases.

What is claimed is:

1. A fusion polypeptide comprising an amino acid sequence corresponding to:
(a) a O-6-alkylguanine-DNA alkyltransferase (AGT) analog, variant and/or a derivative thereof; and
(b) a ribosomal protein or variant, derivative, or fragment thereof.

2. The fusion polypeptide of claim 1, wherein the ribosomal protein is RPL28.

3. The fusion polypeptide of claim 1, wherein the analog, variant and/or a derivative is a SNAP-tag or a CLIP-tag.

4. The fusion polypeptide of claim 3, wherein the SNAP-tag or CLIP-tag comprises an amino acid sequence at least 70% identical to SEQ ID NOS: 1 or 2 respectively.

5. The fusion polypeptide of claim 1, wherein the fusion polypeptide comprises an amino acid sequence at least 70% identical to SEQ ID. NO: 3.

6. A polynucleotide sequence comprising a nucleic acid sequence encoding the fusion polypeptide of any one of claims 1-5.

7. The polynucleotide sequence of claim 6, comprising a nucleic acid sequence at least about 60% identical to SEQ ID NOS: 4 or 5 and further comprising a sequence at least 60% identical to SEQ ID. NO: 6.

8. An engineered host cell comprising a polynucleotide sequence of any one of claim 6 or 7.

9. An engineered host cell comprising a fusion polypeptide comprising an amino acid sequence corresponding to:
(a) an AGT analog, variant and/or a derivative thereof; and
(b) a ribosomal protein or variant, derivative, or fragment thereof.

10. The engineered host cell of claim 9, wherein the ribosomal protein is RPL28.

11. The engineered host cell of claim 9, wherein the analog, variant and/or a derivative is a SNAP-tag or a CLIP-tag.

12. The engineered host cell of claim 11, wherein the SNAP-tag or CLIP-tag comprises an amino acid sequence at least 70% identical to SEQ ID NOS: 1 or 2 respectively.

13. The engineered host cell of claim 9, wherein the fusion polypeptide comprises an amino acid sequence at least 70% identical to SEQ ID. NO: 3.

14. The engineered host cell of any one of claims 8-13, wherein the engineered host cell is a somatic cell or a pluripotent cell.

15. The engineered host cell of any one of claims 8-14, wherein the engineered host cell is a human cell.

16. The engineered host cell of claim 15, wherein the engineered host cell is selected from a HEK293T, HCT116, iPS or hES cell.

17. A method for generating an engineered host cell comprising a SNAP-tagged and/or CLIP-tagged ribosome, the method comprising delivering to a cell:
(a) a first site-directed nuclease cutting at a target site within or near a gene that encodes a ribosomal protein; and
(b) a nucleic acid comprising a nucleotide sequence encoding a SNAP-tagged and/or CLIP-tagged ribosomal protein that is flanked by (i) a nucleotide sequence homologous with a region located upstream of the target site of (a) and (ii) a nucleotide sequence homologous with a region located downstream of the target site of (a),
wherein the first site-directed nuclease cleaves the target site of (a) and the nucleic acid of (b) is inserted at a site that partially overlaps, completely overlaps, or is contained within, the site of (a).

18. The method of claim 17, wherein the ribosomal protein is RPL28.

19. The method of claim 17, wherein the site-directed nuclease is a Cas nuclease.

20. The method of claim 17, wherein the nucleotide sequence homologous with a region located upstream of the target site is at least about 80% identical to SEQ ID NO 7.

21. The method of claim 17, wherein the nucleotide sequence homologous with a region located downstream of the target site is at least about 80% identical to SEQ ID NO: 8.

22. A method of labeling a ribosome in an engineered host cell, the method comprising:
(a) expressing a fusion polypeptide of any one of claims 1-7 in the cell; and
(b) introducing a first substrate molecule in the engineered host cell.

23. The method of claim 22, further comprising introducing a second and/or a third substrate molecule in the cell.

24. A method of heterochronic labeling of one or more ribosomes in an engineered host cell, the method comprising:
(a) expressing a fusion polypeptide of any one of claims 1-7 in the engineered host cell;
(b) introducing a first substrate molecule into the engineered host cell;
(c) introducing a second and/or a third substrate molecule in the engineered host cell, wherein the second and third substrate molecule is different from the first substrate molecule.

25. The method of any one of claims 22-24, wherein (a) comprises genetically modifying the cell to comprise a polynucleotide sequence encoding the fusion polypeptide.

26. The method of claim 25, wherein the engineered host cell is a somatic cell or a pluripotent cell.

27. The method of any one of claim 25 or 26, wherein the engineered host cell is a human cell.

28. The method of claim 27, wherein the human cell is selected from a HEK293T, HCT116, iPS, or hES cell.

29. The method of any one of claims 22-24, wherein the first substrate, the second substrate and the third substrate are cell-permeable substrates.

30. The method of any one of claims 22-24, wherein the first substrate and/or the second substrate and/or the third substrate is selected from a group consisting of group consisting of BG-GLA-NHS, BG-Maleimide, BG-NH2, BG-PEG-NH2, SNAP-Biotin®, SNAP-Cell® 505-Star, SNAP-Cell® Block, SNAP-Cell® Oregon Green®, SNAP-Cell® TMR-Star, SNAP-Cell® 430, SNAP-Cell® 647-SiR, SNAP-Surface® 488, SNAP-Surface® 549, SNAP-Surface® 594, SNAP-Surface® 649, SNAP-Surface® Alexa Fluor® 488, SNAP-Surface® Alexa Fluor® 546, SNAP-Surface® Alexa Fluor® 647, SNAP-Surface® Block, BG-NH2, CLIP-Biotin®, CLIP-Cell® 505-Star, CLIP-Cell® Block, CLIP-Cell® TMR-Star, CLIP-Surface® 488, CLIP-Surface® 547, CLIP-Surface® 547, CLIP-Surface® 647.

31. A method of visualizing ribosomes in a cell, the method comprising:
(a) expressing a fusion polypeptide of any one of claims 1-7 in the cell;
(b) introducing a first substrate molecule into the cell, wherein the first substrate molecule specifically binds to a first ribosome population; and
(c) visualizing the cell using one or more of fluorescence visualization methods.

32. The method of claim 31, further comprising introducing a second or third substrate molecule in the cell.

33. The method of claim 32, wherein the second or third substrate molecule labels a second or third ribosome population.

34. The method of claim 33, wherein the first, the second, and the third ribosome population are heterochronic.

35. The method of any one of claims 31-34, wherein the first and/or the second substrate and/or the third substrate is selected from a group consisting of group consisting of BG-GLA-NHS, BG-Maleimide, BG-NH2, BG-PEG-NH2, SNAP-Biotin®, SNAP-Cell® 505-Star, SNAP-Cell® Block, SNAP-Cell® Oregon Green®, SNAP-Cell® TMR-Star, SNAP-Cell® 430, SNAP-Cell® 647-SIR, SNAP-Surface® 488, SNAP-Surface® 549, SNAP-Surface® 594, SNAP-Surface® 649, SNAP-Surface® Alexa Fluor® 488, SNAP-Surface® Alexa Fluor® 546, SNAP-Surface® Alexa Fluor® 647, SNAP-Surface® Block, BG-NH2, CLIP-Biotin®, CLIP-Cell® 505-Star, CLIP-Cell® Block, CLIP-Cell® TMR-Star, CLIP-Surface® 488, CLIP-Surface® 547, CLIP-Surface® 547, CLIP-Surface® 647.

36. A method of purifying ribosomes from a cell, the method comprising steps of any one of the methods of claims 31-34 and further comprising a ribosome purification step.

37. The method of claim 36, wherein the ribosome purification step comprises use of a SNAP/CLIP-tag-Trap.

38. A kit comprising a polynucleotide of any one of claim 6 or 7 and instructions for use.

39. A kit comprising a polypeptide of any one of claims 1-5 and instructions for use.

40. A kit comprising an engineered host cell of any one of claims 8-16 and instructions for use.

41. The kit of any one of claims 38-40 further comprising suitable substrates, reagents, buffers, diluents, cells, standards, and containers.
